(19) United States
(12) Patent Application Publication
(10) Pub. No.: US 2020/0397732 A1
(43) Pub. Date: Dec. 24, 2020

(54) METHODS AND COMPOSITIONS FOR IDENTIFICATION AND MODULATION OF MICROBIOME BIOCHEMICAL PATHWAYS TO ALTER PHENOTYPE

(71) Applicant: Cybele Microbiome, Inc., San Diego, CA (US)

(72) Inventors: Nicole M. Scott, San Diego, CA (US); Eddie Adams, San Diego, CA (US)

(21) Appl. No.: 16/769,437

(22) PCT Filed: Dec. 4, 2018
(86) PCT No.: PCT/US18/63874

§ 371 Date: Jun. 3, 2020

Related U.S. Application Data
(60) Provisional application No. 62/594,531, filed on Dec. 4, 2017.

(51) Int. Cl. A61K 31/20; A61K 9/00; A61K 31/03; A61K 31/11; A61K 31/015

(52) U.S. Cl. CPC A61K 31/20; A61K 9/0014; A61K 31/015; A61K 31/11; A61K 31/03

(57) ABSTRACT

Provided, in part, are methods, compositions, and systems are provided for detecting one or more repellent-generating enzymatic pathways by characterizing the skin microbiomes and metabolomes of populations of individuals both naturally resistant and highly prone to bites from blood sucking arthropods, determining the microbial taxa (and their associated metabolic pathways) most associated with insect repellency and targeting/activating those pathways with molecular substrates that, when metabolized, will give rise, in situ, to repellent molecules and can promote a shift to a more naturally repellent microbiome composition.

### FIELD

Provided herein, at least in part, are methods for identifying microbiome pathways associated with a phenotype. Also provided are methods and compositions for the identification and generation of insect repellents of dermal microbial origin. In some embodiments the compositions include substrates that, when metabolized, will generate repellent molecules.

### BACKGROUND

N,N-Diethyl-3-methylbenzamide (DEET) is the most common mosquito repellent since its synthesis in 1944. While other synthetic agents, such as picaridin and IR3535 have come into use alongside DEET, there has been considerable consumer interest in finding repellents derived from natural sources like plants. These include essential oils such as citronella, rosemary oil, cinnamon oil, mint oil, clove oil, catnap oil, and lemon eucalyptus oil.

What all of these agents—wholly synthetic and plant-derived—have in common is that they are not derived from the skin of the subjects they are meant to protect. That is to say, they are both exogenously derived, and applied often at very high concentrations in order to provide sufficient strength and duration of insect repellency. These high concentrations give rise to textures, odors, and in some cases, skin irritation that are undesirable to the user. Furthermore, the required high concentrations of these agents often prevent their incorporation into products that provide other beneficial properties, such as sunscreens. Furthermore, arthropods can develop resistance to single agent repellent formulations.

It has long been appreciated that certain individuals in the population are less prone to mosquito bites than others. It has also been appreciated that of the various stimuli—visual, thermal, moisture, and chemical—involved in attracting female mosquitoes to a host, chemical cues are likely the most important. Chauhan and Bernier (2015) report estimates of 300-400 compounds released from the human body as by-products of metabolism, with at least 100 compounds being detected in the breath. These various chemical emanations allow ranking human subjects for their attractiveness to mosquitoes according to the subjects' chemical emanations.

Skin microbes play a key role in the generation of body odor and volatile generation. Verhulst et al. employed 454 pyrosequencing to identify, via 16S rRNA sequencing, the bacterial genera most strongly associated with human subjects whose skin emanations were either poorly attractive or highly attractive to the mosquito Anopheles gambiae. This analysis provided an incomplete picture of the genetic networks involved in creating a poorly attractive metabolome. Not only was this study limited to the analysis of bacteria (no analysis of fungi was performed, for example), it is unclear how one would catalog molecules that may serve as bacterially-derived repellents, nor how to modulate the skin microbiome to create a more naturally repellent skin phenotype.

Companion animals are considered a reservoir for many vector borne diseases, meaning that either they are directly zoonotic (being transmittable between animals and humans) or have the potential (for example via a genetic mutation) for being transmittable. Because of this potential, companion animals have become of increasing interest in the public health of vector borne diseases.

### SUMMARY

Provided herein, at least in part, are methods and systems of identifying phenotype-generating biochemical pathways from metagenomic sequencing and metabolome profiling. Disclosed herein are compounds that, when metabolized, can serve to alter a microbiome or meta-transcriptome composition by driving the preferential growth or transcriptional activity of those phenotype-generating microbial species capable of acting on the compound.

Some of the embodiments provided herein relate to methods for identifying one or more biochemical pathways associated with a phenotype of a dermal sample that include determining a first metabolome compound matrix of a dermal sample of at least one subject lacking a phenotype, determining a second metabolome compound matrix of a dermal sample of at least one subject possessing a phenotype, comparing the first and second metabolome compound matrices, and associating differences between the first and second metabolome compound matrices to at least one biochemical pathway; wherein the at least one biochemical pathway is associated with the phenotype. In some embodiments the phenotype includes insect repellence. In some embodiments the phenotype includes insect attraction. In some embodiments the phenotype includes a dermal metabolite. In some embodiments the dermal sample includes skin, hair, or fur. In some embodiments the method further includes identifying at least one agent that is a compound of the at least one biochemical pathway. In some embodiments the determining of the first or second metabolome compound matrix includes an analytical method including: a genomics method assessment, a transcriptomic or metabolomics assessment, a determination of microbiome composition, nuclear magnetic resonance (NMR) and mass spectrometry (MS), Fourier-transform infrared (FTIR), infrared (IR) thermography, cataluminescence (CTL), laser-induced fluorescence imaging (LIFT), and resonance-enhanced multiphoton ionization (REMPI). In some embodiments the determining of the first or second metabolome compound matrix is obtained from one or a plurality of subjects.

Some embodiments provided herein relate to methods for identifying one or more biochemical pathways associated with a phenotype of a dermal sample that include determining a first metagenome matrix of a dermal sample of at least one subject lacking a phenotype, determining a second metagenome of a dermal sample of at least one subject possessing a phenotype, comparing the first and second metagenome matrices, and associating differences between the first and second metagenome matrices to at least one biochemical pathway of the subject wherein the at least one biochemical pathway is associated with the phenotype. In some embodiments the phenotype includes insect repellence or insect attraction. In some embodiments the phenotype includes a dermal metabolite. In some embodiments the dermal sample includes skin, hair, or fur. In some embodiments the method further includes identifying at least one agent that is a compound of the at least one biochemical pathway. In some embodiments the determining of the first or second metagenome matrix is obtained from one or a plurality of subjects. In some embodiments the metagenome matrix includes a metatranscriptome. In some embodiments the method further includes identifying at least one agent that modulates the gene expression of at least one component for the at least one biochemical pathway.

Some embodiments provided herein relate to methods of modulating a phenotype of a subject by contacting the subject with a compound identified by a method described above. In some embodiments the subject lacks the phenotype. In some embodiments the subject possesses the phenotype. In some embodiments the phenotype includes insect repellence or insect attraction.

Some embodiments provided herein relate to methods for identifying one or more biochemical pathways associated with a phenotype of a gut sample including determining a first metabolome compound matrix of a gut sample of at least one subject lacking a phenotype, determining a second metabolome compound matrix of a gut sample of at least one subject possessing a phenotype, comparing the first and second metabolome compound matrices and associating differences between the first and second metabolome compound matrices to at least one biochemical pathway, wherein the at least one biochemical pathway is associated with the phenotype. In some embodiments the phenotype includes insect repellence. In some embodiments the phenotype includes insect attraction. In some embodiments the phenotype includes a gut metabolite. In some embodiments the gut sample includes esophagus, stomach, small intestine, large intestine, or a fecal material. In some embodiments, the gut sample includes fecal material. In some embodiments the method further includes identifying at least one agent that is a compound of the at least one biochemical pathway. In some embodiments the determining of the first or second metabolome compound matrix includes subjecting the gut sample to an analytical method selected from the group consisting of: a genomics method assessment, a transcriptomic or metabolomics assessment, a determination of microbiome composition, nuclear magnetic resonance (NMR) and mass spectrometry (MS), Fourier-transform infrared (FTIR), infrared (IR) thermography, cataluminescence (CTL), laser-induced fluorescence imaging (LIFT), and resonance-enhanced multiphoton ionization (REMPI). In some embodiments the determining of the first or second metabolome compound matrix is obtained from one or a plurality of subjects.

Some embodiments provided herein relate to methods for identifying one or more biochemical pathways associated with a phenotype of a gut sample including determining a first metagenome matrix of a gut sample of at least one subject lacking a phenotype, determining a second metagenome of a gut sample of at least one subject possessing a phenotype, comparing the first and second metagenome matrices, and associating differences between the first and second metagenome matrices to at least one biochemical pathway of the subject wherein the at least one biochemical pathway is associated with the phenotype. In some embodiments the phenotype includes insect repellence. In some embodiments the phenotype includes insect attraction. In some embodiments the phenotype includes a gut metabolite. In some embodiments the gut sample includes esophagus, stomach, small intestine, large intestine, or a fecal material. In some embodiments the method further includes identifying at least one agent that is a compound of the at least one biochemical pathway. In some embodiments the determining of the first or second metagenome matrix is obtained from one or a plurality of subjects. In some embodiments the metagenome matrix includes a metatranscriptome. In some embodiments the method further includes identifying at least one agent that modulates the gene expression of at least one component for the at least one biochemical pathway.

Certain embodiments are described further in the following description, examples, claims, and drawings.

### BRIEF DESCRIPTION OF THE DRAWINGS

The drawings illustrate certain embodiments of the technology and are not limiting. For clarity and ease of illustration, the drawings are not made to scale and in some instances, various aspects may be shown exaggerated or enlarged to facilitate an understanding of particular embodiments.

FIG. 1 shows Principal Coordinates analysis of scores showing clustering of individuals that were phenotyped as attractive (shown here in light grey) and those that were unattractive (shown here in dark) to mosquitos.

FIGS. 2A-2B shows an example of predicted compounds highlighted (in dark) on portions of their associated KEGG pathway. FIG. 2B is a continuation of the pathway shown in FIG. 2A.

FIGS. 3A-3B schematically depicts the top most accumulated metabolites and their respective pathways related to repellency.

FIGS. 4A-4B schematically depicts the top most consumed metabolites and their respective pathways related to repellency.

FIGS. 5A-5B schematically depicts the top most accumulated metabolites and their respective pathways related to oral health.

FIGS. 6A-6B schematically depicts the top most consumed metabolites and their respective pathways related to oral health.

FIGS. 7A-7B schematically depicts the top most consumed metabolites and their respective pathways related to atopic dermatitis.

FIGS. 8A-8B schematically depicts the top most accumulated metabolites and their respective pathways related to atopic dermatitis.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the figures, can be arranged, substituted, combined, separated, and designed in a wide variety of different configurations, all of which are explicitly contemplated herein.

A microbiome is a population of microorganisms within a specified environment. Animals may have multiple distinct microbiomes, including, for example, the gut microbiome, the skin microbiome, the lung microbiome, and the oral microbiome.

The skin microbiome includes a richly diverse population of organisms. These organisms produce various compounds by a complex array of biochemical pathways, which result in health of the skin, general health and well-being, and emanation of odors. In addition, the skin microbiome can result in attractiveness or repellency of various undesirable insects, such as mosquitos.

Provided herein are systems and methods for modulating a microbiome, and in particular, for modulating the biochemical machinery of the microbiome. The microbiome that may be modulated may be any particular microbiome of interest, for example, the gut microbiome, the skin microbiome, the oral microbiome, the vaginal microbiome, the ocular microbiome, or the lung microbiome, or other microbiomes of interest, or any combination thereof. The systems and methods described herein may have numerous applications related to modulation of a microbiome. Embodiments provided herein relate to methods and systems to determine the compounds necessary to enhance the microbiome. The methods and systems provided herein relate to actively stimulating metabolic pathways within the natural microbiome. The general platform described herein has many applications. One such example is insect repellency, which is described herein in detail by way of example. However, a person of skill in the art will recognize that insect repellency serves as an exemplary application, and that the general platform described herein relates to additional practical applications. In addition, the general platform is described herein with reference to the skin microbiome, but it is to be understood that the same principles may be used with respect to other microbiomes.

Accordingly, provided herein are systems and methods for modulating a microbiome, including a gut microbiome, a skin microbiome, an oral microbiome, a lung microbiome, or other microbiome, or any combination thereof. Modulation of a microbiome may enhance a certain desirable phenotype, for example, enhance gut health (including, for example, improving gut metabolism, boosting immune function, or improving digestive health and nutritional uptake), skin health (including, for example, enhancing, changing, or reducing body odor, repellency, or overall skin health or improving atopic dermatitis), oral health (including, for example, improving or preventing caries or other periodontal disease), lung health (including, for example, ameliorating or preventing lung disease, such as chronic obstructive pulmonary disease), or overall health and well-being. Furthermore, the systems and methods described herein may be used in some embodiments for the production of microbiome-generated products, for use, for example, in cosmetics.

In some embodiments, the methods and systems described herein for modulating a microbiome are used for improving repellency. Many individuals highly attract mosquitoes, resulting in agitating, itchy, and painful welts that can subsist for days or even weeks. Other individuals are naturally unattractive to mosquitoes and even produce substances that actively repel mosquitoes. This differential attraction to insects is explained by the chemistry on a skin surface that generates this difference in attraction to mosquitoes. The chemistry on the skin is created by organisms, including bacteria, single-celled organisms, and viruses, collectively referred to as the skin microbiome. The microbiome, and not the CO₂, lactic acid, or blood type, creates differences in attraction to mosquitoes. In fact, human sweat is odorless without processing by the skin microbiome.

In addition to being a source of physical irritation, mosquitoes are also deadly carriers of disease. For example, mosquitoes are known to carry the Zika virus, bird flu, yellow fever, malaria, Chikungunya virus, West Nile virus, dengue, and other illnesses. These illnesses affect hundreds of millions of people around the world and cause millions of deaths each year. Despite mosquito repellent products, such as DEET, disease rates continue to rise. This is a result both from individuals being less willing or able to use available products because of their cost, perceived toxicity, feeling on the skin, or ineffectiveness, as well as decreased susceptibility of the mosquitos to products.

However, by way of example, one such application of enhancing skin microbiome would be for stimulating natural repellency. In this example, the methods and compositions described herein enhance functional pathways within the skin microbiome to create a repellency that is carried with an individual. Whereas conventional repellent products applied to the skin that have the active ingredients within in the product that last at most for hours, the methods and systems stimulate metabolic pathways within the natural microbiome. This results in a natural, long-lasting, and effective mosquito repellent. The result is a non-toxic natural repellency that is safe for daily use by any individual, including mothers, children, pregnant women, and travelers. In addition, this results in a natural product that avoids environmental issues, toxicity, and insect resistance of DEET or other chemical repellents.

Some embodiments provided herein relate to methods, compositions, and systems for detecting one or more phenotype-generating enzymatic pathways by comparing the skin or gut microbiomes and metabolomes of one or more subjects with a phenotype and those without. Also provided are methods for determining the microbial taxa or a representation of those organisms (and their associated metabolic pathways) most associated with a phenotype. Also provided are compositions and methods for modulating the phenotype of a subject by contacting the subject with one or more compounds that promote a shift to or from a phenotype.

Methods, compositions, and systems are provided for detecting one or more repellent-generating biochemical pathways by characterizing the skin microbiomes or metabolomes of populations of individuals both naturally resistant and highly prone to bites from blood sucking arthropods. Provided also are methods for determining the microbial taxa (and their associated metabolic pathways) most associated with insect repellency. In other embodiments, provided are methods and compositions for targeting/activating biochemical pathways with molecular substrates that, when metabolized, will give rise, in situ, to repellent molecules and can promote a shift to a more naturally repellent microbiome composition.

As used herein, “microbiome” means the combined genetic material of the microorganisms in a sample. A microbiome can include microorganisms, both living and nonliving; bacteria, archaea, viruses, and eukaryotes. As used herein, a “representation of a microbiome” is the data associated with a microbiome. The representation of a microbiome can include, but is not limited to, genetic markers of microorganisms; metabolic markers of microorganisms; genetic markers of metabolic markers; and combinations thereof. Microbiome containing samples and representations of microbiomes may be acquired from public sources (in some embodiments: the Human Microbiome Project, NCBI, Earth Microbiome Project), private sources, local collection, real-time collection, or historical collection.

As used herein, the term “metagenome” means all the genetic material in a sample of a plurality of organisms. A metagenome can include: microorganisms, both living and nonliving; bacteria, archaea, viruses, and eukaryotes; genetic markers of organisms; genetic markers of metabolic markers; and combinations thereof. Such samples may be acquired from public sources (in some embodiments: the Human Microbiome Project, NCBI, Earth Microbiome Project), private sources, local collection, real-time collection, or historical collection. As used herein, plurality refers to one or more than one organisms. Thus, where a method refers to obtaining a matrix, for example, from a plurality of subjects, the method may include one subject, or many subjects. In some embodiments, a 1:1 sample comparison is performed.

As used herein, the term “metagenome matrix” means a dataset of the counts of nucleic acids in a sample of a plurality of organisms.

As used herein, “gene community composition” includes annotations of genetic material present, it can include the abundances of this material. A gene community composition may be from a single sample or many samples.

As used herein, “community composition” means the identity of the organisms present, although it may also include the abundances of those organisms present. A community composition may represent a single sample or many samples. Further, the community composition may be genetic features or genetic data. Data can be from public databases or datasets, or estimated from other parameters.

As used herein, the term “metabolome compound matrix” means a dataset of the counts of enzyme or reactive causative compounds involved in biochemical reactions and the associated compounds that are considered the products or reactants for that reaction. In some embodiments, the counts are the times the reactive causative compounds are seen with a particular product or reactant, where the times it is seen with products are considered to be positive and the times it is seen with the reactants is negative. Biochemical pathways or reactions can come from a host of sources including in some embodiments: public databases, curated data, empirical observations, experimental work or a combination thereof. The data can also be estimated or hypothesized. The matrix is one representation of a metabolome.

As used herein, the term “metabolome” means the complete set of chemicals found within a biological sample. The biological sample can be a cell, a cellular organelle, an organ, a tissue, a tissue extract, a biofluid or an entire organism. In some embodiments, the biological sample is skin.

As used herein, the term “metabolite” means the chemical intermediates or products of metabolism. In some embodiments, metabolites are small molecules. In some embodiments, metabolites include polymeric biomolecules such as DNA, RNA or proteins greater than 100 amino acids in length. A metabolite can be a substrate for an enzyme of a metabolic pathway, an intermediate of such a pathway or the product obtained by the metabolic pathway.

As used herein, the term “metadata” means data that describes other data. Metadata can include data associated with the sample environment, a state of interest, active inquiry, or status. For example, that the data has assigned to each sample a ‘state’ that is being investigated. Otherwise, data for which there is no state associated with it, may be predicted, but only on the basis of some sort of database in which part or all of the samples' status is known.

As used herein, the term “metatranscriptome” means the complete or partial repertoire of RNA transcripts produced by a plurality of microbes inhabiting the skin or other environment, and may be represented by DNA.

As used herein, the term “repellent” means an agent that causes insects to make oriented movements away from the agent's source. In some embodiments, using the EPA's test guidelines, a repellent is also defined as “a product intended to disrupt the host-seeking behavior of insects or other arthropods, driving or keeping them away from treated human skin.”

As used herein, the term “arthropod” refers to an invertebrate animal having an exoskeleton, and that perturbs a host. For example, an arthropod may be a mosquito, fly, tick, flee, mite, louse, bed bug, spider or any number of numerous related insects that are desirable to repel or prevent contact with.

As used herein, the term “attractant” means an agent that causes insects to make oriented movements toward the agent's source.

In some embodiments the method further includes identifying at least one agent that is a compound of the at least one biochemical pathway. In some embodiments the determining of the first or second metabolome compound matrix includes subjecting the gut sample to an analytical method selected from the group consisting of: a genomics method assessment, a transcriptomic or metabolomics assessment, a determination of microbiome composition, nuclear magnetic resonance (NMR) and mass spectrometry (MS), Fourier-transform infrared (FTIR), infrared (IR) thermography, cataluminescence (CTL), laser-induced fluorescence imaging (LIFT), and resonance-enhanced multiphoton ionization (REMPI). A genomics method assessment may include any method for analyzing genomics, including, for example, sequencing, nucleic acid analysis, ribonucleic acid analysis, and the like.

“Skin”, as used herein, means the outermost layer of a subject body and can include the hair and other debris that is present; the epidermis or dermis; the covering of the animal body; fur, or hair. A “dermal sample” is a skin specimen. As used herein, the term “subject” is an animal, such as a vertebrate, including a mammal. The term “mammal” is defined as an individual belonging to the class Mammalia and includes, without limitation, humans, domestic and farm animals, and zoo, sports, or pet animals (companion animals), such as sheep, dogs, horses, cats or cows. Suitable subjects include laboratory animals (such as mouse, rat, rabbit, or guinea pig), farm animals, and domestic animals or pets (such as a cat or dog). Non-human primates or, preferably, human patients, are included.

As used herein, the term “phenotype” means one or more observable characteristics of an individual resulting from the interaction of its genotype with the environment. The phenotype can include metadata, parameters, conditions, and physical designation. Phenotypes can include insect repellence or insect attraction.

As used herein, the term “biochemical pathway” means the chemical reactions involved in operations of organisms.

Provided herein are methods for identifying one or more biochemical pathways associated with a phenotype of a biological sample. In some embodiments, the biological sample is a dermal sample. A dermal sample can include skin, hair or fur. A dermal sample can be obtained from a specified body site. A specified body site can be, for example, an ankle, a forearm, an underarm, a nipple area, or a neck. In some embodiments, the biological sample is a gut sample. The gut sample can include, but not limited to, a sample from the esophagus, stomach, small intestine, large intestine, or from a fecal material. A phenotype can be insect repellence or insect attraction.

A first metabolome compound matrix is determined for a biological sample of at least one subject lacking a phenotype. A second metabolome compound matrix is determined for a biological sample of at least one subject possessing a phenotype. The first and second metabolome compound matrices are compared and one or more differences are associated with at least one biochemical pathway, thereby identifying the at least one biochemical pathway as associated with the phenotype.

In some embodiments, the methods are useful for identifying skin microbiome metabolic pathways associated with a phenotype. The methods are also useful for identifying endogenous phenotype-associated molecules derived from the skin microbiome. These methods can include performing mass spectrometry on biological samples that include skin emanations from distinct body sites from subjects who have the phenotype of interest to biological samples that include skin emanations from the same distinct body sites from subjects without the phenotype. The data relating to either set of subjects (with or without the phenotype) can be obtained from a database.

Also provided are methods for determining the biochemical pathways that generate a phenotype associated with certain skin emanations. The methods identify biochemical pathways operative to generate the molecules present in the skin emanations from a population of individuals by comparing a phenotype-possessing population to a phenotype-lacking population.

Also provided are methods for identifying enzyme substrates that, when metabolized by a host microbiome, results in a skin microbiome desired phenotype molecules. These methods identify biochemical pathways are operative to generate a phenotype and identifying substrate molecules for an operative biochemical pathway's enzymes.

Skin is a heterogeneous surface, for example, with folds, differences in temperatures and humidity. The microbiome that exists in different body sites are thereby also different. Sweat glands (apocrine and eccrine) have higher density in various body sites. For example, apocrine glands that are located in armpit, urogenital, and nipple regions excrete odorless substances. The secretions are metabolized by microbes on the host body to create the odor associated with sweat. The microbial communities are commensal, living in concert with host excretions and immune system. The host recognizes the microbial community as a non-threat via a number of different receptors in keratinocytes located in the epidermis. Pattern recognition receptors (PRR) recognize different pathogen-associated molecular patterns (PAMPs) including flagellin, nucleic acids, lipopolysaccharides on the surface of Gram-negative bacteria, peptidoglycan and lipoteichoic acid on the surface of Gram-positive bacteria, and zymosan and mannan from fungal cell walls. When PRRs are activated by PAMPs, the host excretes cytokines, chemokines and antimicrobial peptides. PRRs include toll-like receptors, mannose receptors, and NOD-like receptors (Grice et al. 2008). Human Leukocyte Antigen (HLA) types are associated with attractiveness to mosquitos, and certain compounds are associated with either attractiveness or unattractive phenotypes (Verhulst et al. 2013).

Also provided herein are methods and systems of identifying repellent-generating biochemical pathways from whole genome metagenomic sequencing and metabolome profiling. The identified biochemical pathways are used to catalog compounds that, when metabolized, can form repellent molecules in situ. In addition, the compound can also alter the skin microbiome or meta-transcriptome composition by driving the growth or transcriptional activity of repellent-generating microbial species capable of acting on the provided compounds. Also, in some embodiments, the compound can also alter the skin microbiome or meta-transcriptome composition by reducing the growth or transcriptional activity of attractant-generating microbial species capable of acting on the provided compounds.

Also provided are methods for identifying skin microbiome metabolic pathways associated with endogenous arthropod repellency. Metagenomes of dermal samples from one or more body sites from a population of subjects whose skin emanations, from those same body sites, are poorly attractive to an arthropod of interest are determined by whole genome sequencing, or by another means of identifying organisms in samples.

Also provided are methods for identifying endogenous arthropod repellent molecules derived from the skin microbiome. This method comprises performing mass spectrometry of dermal samples containing skin emanations from body sites from a population of subjects whose skin emanations, from those same body sites, are poorly attractive to the arthropod of interest.

Also provided are methods for determining one or more biochemical pathways that generate arthropod-repellent skin emanations. In some embodiments, the methods comprise determining which biochemical pathways are operative to generate the molecules present in the skin emanations from a population of subjects whose skin emanations are poorly attractive to the arthropod of interest.

Also provided are methods for identifying enzyme substrates that, when introduced to a subject, can result in skin microbiome derived arthropod-repellent molecules. In some embodiments, the methods comprise determining which biochemical pathways are operative to generate arthropod-repellent skin emanations and identifying at least one substrate molecule for an operative biochemical pathway's enzyme.

Arthropods that transmit such disease include mosquitos, ticks, fleas, and sandflies, and lice among others. Vector borne diseases can be avoided by preventing the arthropod vectors from feeding on the agent, the companion animal, or any reservoirs. Control of vectors can be accomplished in a number of ways including environmentally, contact reduction, chemically, and biologically. Methods can also be combined, used in concert and supplemented to control vectors. Some embodiments could also be combined with any of these techniques. Those described here are not intended to be an inclusive or exhaustive list.

The metabolome compound community (M) represents functional or putatively functional gene pathways. M includes the conditional probability counts of enzymes actively transforming, catalyzing processes and their reactants and metabolites. For example, it is possible to predict regions of enzymatic activity by algorithmic and empirical approaches, and it is also possible via annotation methods. For example, Table 1 shows a set of reactions used for a metabolome compound matrix:

TABLE 1

| Pathway | Reactant | Enzyme | Product |
|---|---|---|---|
| 362 | CO2 | 2.7.1.105 | Pyruvate |
| 660 | Pyruvate | 1.1.1.— | CO2 |
| 630 | CO2 | 5.1.3.20 | 2-Hydroxy-3- |
| | | | oxopropanoate |
| 10 | Urea-1- | 2.2.1.6 | 2-(alpha-Hydroxy- |
| | carboxylate | | ethyl)thiamine |
| | | | diphosphate |
| 10 | Pyruvate | 2.2.1.6 | 2-(alpha-Hydroxy- |
| | | | ethyl)thiamine |
| | | | diphosphate |

Table 2 shows the resulting estimated metabolome compound community:

TABLE 2

| | Compound | | | | |
|---|---|---|---|---|---|
| | | | | | 2-(alpha-Hydroxy- |
| | | | Urea-1- | 2-Hydroxy-3- | ethyl)thiamine |
| Enzyme | CO2 | Pyruvate | carboxylate | oxopropanoate | diphosphate |
| 2.7.1.105 | −1 | 1 | 0 | 0 | 0 |
| 1.1.1.— | 1 | −1 | 0 | 0 | 0 |
| 5.1.3.20 | −1 | 0 | 0 | 1 | 0 |
| 2.2.1.6 | 0 | −1 | −1 | 0 | 2 |

In some embodiments, samples can be obtained through a multitude of sample collection methods. Samples may be collected through direct assessment, public data, or even estimated from other parameters. It can be obtained by metagenomic sequencing. A gene community composition matrix (G) contains the counts of genes related to enzymes or catalyzing processes in the metabolome compound community, likewise it can represent sequence regions. For example, a partial gene community composition matrix is shown in Table 3, where it has been log 2 scaled and quantile normalized.

TABLE 3

| | v59D13.5.08 | v55D11.12.07 | v33D19.3.08 | v52D1.2.08 |
|---|---|---|---|---|
| 2.7.1.105 | 12.335644 | 12.509551 | 12.509551 | 12.631521 |
| 1.1.1.— | 3.373017 | 5.721894 | 4.238183 | 7.327579 |
| 5.1.3.20 | 10.585727 | 10.961677 | 11.419513 | 11.201647 |
| 2.2.1.6 | 9.235792 | 8.811617 | 9.316150 | 9.353249 |

The gene community composition is then transformed by the metabolome compound matrix, such that a score is produced. The transformation can be a dot product of the gene community matrix with the metabolome compound matrix. Scores then represent the putative transformation of the compound given that gene community composition. For example, assuming gene community composition per sample, the result is a set of metabolites or compounds within a sample each with a score.

Provided herein are methods that predict organism to metabolite relationship. In this embodiment, G is normalized, as before but G is then scaled by the proportion of organisms holding those genes. For example here, assuming is a gene by sample matrix, e.g. that many samples are contained by G, we scale G by the per sample per organism gene matrix, where each sample is a vector of the proportional counts within a sample here denoted as \vec{s}ₒ. The score then becomes, M=( [symbol] )=sₒ; the resulting scores are partitioned by organism in a sample.

The scores can be positive or negative representing either accumulation or increased turnover, depending on their use and how scores are processed. Such representation of the community activity can be visualized by taking the pairwise distances between samples and displaying them in N-dimensional space. Generally, distance matrices of the scores can show inter-relationships between samples in N-dimensional space and group differences. Likewise, scores can be averaged within a status group (in other words by associated metadata) and then subtracted, to determine the group in which increased or decreased transformation or accumulation is occurring. Scores can also be correlated with metadata. Further, using public, private, empirical, sample or curated pathways, or some combination of these, compounds can be annotated or aggregated. P-values of scores are found by permuting the final scores to find the null distribution and comparing the occurrence of the score to the percentiles of the empirical null distribution. Scores may be used on their own or in accompaniment with the following to determine community changes based on optimizing the set of values.

Some embodiments also include estimation of the physical amount values of these compounds. Compound values can also be mass estimated by incorporating metabolomics data including, but not limited to, Fourier transform ion cyclotron resonance mass spectrometry (FT-ICR/MS). Such methods can be used to calculate the mass for a compound, and in turn an estimate of the abundance mass (a) for a compound in a sample can be calculated as, for example

$$\frac{m \text{?}}{w \text{?}} M * G = {S}_{A} , \\ \text{?} \text{indicates text missing or illegible when filed}$$

where the vector m is the masses of compounds via the metabolome, w is the molecular mass per compound, M is the metabolome matrix and G is the gene community composition and S_{A} is the mass adjusted compound score matrix. These scores can be visualized in a number of meaningful ways such as those given above for the unadjusted scores.

Local microenvironment affects microbial community composition. Available nutrients, temperature, salinity, pH, host, and others, are all factors that shape the types of organisms in a niche. Community composition can also be dynamic, but an overview of the processes occurring, such as in some embodiments, allow the important compounds for shifting organisms that are also associated to the status, metadata, environment, to be discerned. Metabolomics data can be obtained by methods known in the art including, but not limited to, nuclear magnetic resonance (NMR) and mass spectrometry (MS), Fourier-transform infrared (FTIR), infrared (IR) thermography, cataluminescence (CTL), laser-induced fluorescence imaging (LIFT), or resonance-enhanced multiphoton ionization (REMPI). Using the methods disclosed herein can identify the organisms, the compounds necessary, and potential volumes of each. The data can also be used directly to determine the amounts present in a sample.

Also provided are methods for selectively dosing microbiome metabolic pathways to generate molecules that affect the phenotype of interest. These methods include selecting one or more microbial enzyme substrates that, when metabolized, will form molecule(s) that create the phenotype of interest.

In some embodiments, methods are provided for inducing changes in the metatranscriptome of the skin microbiome to drive the generation of a skin environment of interest. This method comprises dosing one or more metagenomic pathways with substrate molecules to affect the upregulation of the requisite metabolic machinery to process said substrate molecules and in some cases, the downregulation of non-requisite metabolic machinery or negative regulators of the requisite pathway components.

In other embodiments, the methods include inducing changes in the metabolome formed by the skin microbiome to one of a skin environment of interest. This method comprises dosing one or more metagenomic enzyme pathways with substrate molecules to affect the metabolic conversion of the supplied substrate molecules to phenotype-inducing molecules.

In other embodiments, the methods include shifting the skin microbiome away from one associated with a phenotype to one that is not associated. This method comprises dosing one or more pathways with phenotype-generating substrate molecules with sufficient regularity to induce the preferential outgrowth of microbial species capable of metabolizing the dosed molecules.

In other embodiments, the methods include shifting the gut microbiome away from one associated with a phenotype to one that is not associated. This method comprises dosing one or more pathways with phenotype-generating substrate molecules with sufficient regularity to induce the preferential outgrowth of microbial species capable of metabolizing the dosed molecules. This can be used in concert with skin methods described here.

Also provided are methods for contacting a subject with at least one compound wherein the microbiome generates arthropod-repellent molecules. In some embodiments, the at least one compound is identified by methods disclosed above. This method includes selecting one or more microbial enzyme substrates that, when metabolized, will form arthropod-repellent molecule(s). In some embodiments, the at least one compound modulates the metatranscriptome of the skin microbiome of a subject to modulate the arthropod-repellent skin environment. In some embodiments, the at least one compound modulates the metatranscriptome of the gut microbiome of a subject to modulate the arthropod-repellent gut environment. In some embodiments, the at least one compound modulate the regulation of the requisite metabolic machinery to process said substrate molecules and in some cases, the downregulation of non-requisite metabolic machinery or negative regulators of the requisite pathway components. In some embodiments, the at least one compound changes in the metabolome formed by the skin microbiome to one of an arthropod-repellent skin environment. In some embodiments, the at least one compound changes in the metabolome formed by the gut microbiome to one of an arthropod-repellent gut environment. In some embodiments, the at least one compound results in volatile, arthropod-repellent molecules. In some embodiments, the skin microbiome is shifted from one associated with arthropod attractiveness to one that is arthropod repellent. In some embodiments, the gut microbiome is shifted from one associated with arthropod attractiveness to one that is arthropod repellent. In some embodiments, the subject is contacted with the at least one compound at a dosing schedule sufficient to induce the preferential outgrowth of microbial species capable of metabolizing the at least one compound.

For compounds that could be used shift microbiome towards insect repellency or create a microbiome that aids or acts in concert with the skin in repellency, would include, but are not limited to, the following alone or in combination from those in the following biochemical pathways: Xylene degradation; Glucosinolate biosynthesis; Aminoacyl-tRNA biosynthesis; Penicillin and cephalosporin biosynthesis; Valine degradation; Leucine degradation; Isoleucine degradation; Tryptophan metabolism; Steroid biosynthesis; Starch and sucrose metabolism; Pyrimidine metabolism; Purine metabolism; Propanoate metabolism; Butanoate metabolism; Citrate cycle; Tyrosine metabolism; Phenylalanine metabolism; caprolactam degradation or metabolism; Toluene degradation; Glyoxylate metabolism; Dicarboxylate metabolism; Alanine metabolism; Aspartate metabolism; Glutamate metabolism; Propanoate metabolism; Porphyrin metabolism; Chlorophyll metabolism; Polycyclic aromatic hydrocarbon degradation; Benzoate degradation; Phosphonate metabolism; Phosphinate metabolism; Peptidoglycan biosynthesis; Penicillin and cephalosporin biosynthesis; Pantothenate and CoA biosynthesis; Nitrogen metabolism; Cyanoamino acid metabolism; Nicotinate and nicotinamide metabolism; Naphthalene degradation; Methane metabolism; Glycine metabolism; serine metabolism; threonine metabolism; Lysine degradation; Lipopolysaccharide biosynthesis; Inositol phosphate metabolism; Indole alkaloid biosynthesis; Histidine metabolism; Glycerolipid metabolism; Glycerophospholipid metabolism; Phosphatidylinositol signaling system; Fluorobenzoate degradation; Fatty acid biosynthesis; Dioxin degradation; Chlorocyclohexane and chlorobenzene degradation; Butanoate metabolism; Fatty acid metabolism; Arachidonic acid metabolism; Amino sugar metabolism; nucleotide sugar metabolism; vitamin B6 metabolism; geraniol degradation; cintronella degradation; limonene degradation; and pinene degradation.

Compounds to create repellent products would include, but are not limited to, the following alone or in combination from those in the following biochemical pathways: Xylene degradation; Valine, leucine and isoleucine degradation; Terpenoid backbone biosynthesis; Taurine and hypotaurine metabolism; Cysteine and methionine metabolism; Styrene degradation; Tyrosine metabolism; Streptomycin biosynthesis; Polyketide sugar unit biosynthesis; Steroid biosynthesis; Starch and sucrose metabolism; Pyrimidine metabolism; Purine metabolism; Propanoate metabolism; Porphyrin and chlorophyll metabolism; Polycyclic aromatic hydrocarbon degradation; Peptidoglycan biosynthesis; Nicotinate and nicotinamide metabolism; Naphthalene degradation; Purine metabolism; Puromycin biosynthesis; Methane metabolism; Lysine degradation; Isoquinoline alkaloid biosynthesis; Indole alkaloid biosynthesis; Monoterpenoid biosynthesis; Inositol phosphate metabolism; Histidine metabolism; Phosphatidylinositol signaling system; Glycerolipid metabolism; Glycerophospholipid metabolism; Glutathione metabolism; Geraniol degradation; Fluorobenzoate degradation; Fatty acid biosynthesis; Drug metabolism—other enzymes; Dioxin degradation; D-Alanine metabolism; Peptidoglycan biosynthesis; Aminoacyl-tRNA biosynthesis; One carbon pool by folate; glyoxylate metabolism; dicarboxylate metabolism; Caprolactam degradation; Butanoate metabolism; Brassinosteroid biosynthesis; Biosynthesis of unsaturated fatty acids; Biosynthesis of type II polyketide backbone; Tetracycline biosynthesis; Benzoate degradation; Tryptophan metabolism; Dioxin degradation; Arginine metabolism; Proline metabolism; Arachidonic acid metabolism; Aminoacyl-tRNA biosynthesis; Valine biosynthesis; Leucine biosynthesis; Isoleucine biosynthesis; Amino sugar metabolism; and nucleotide sugar metabolism.

The systems and methods described herein may be used for modulation of various microbiomes, including, for example, the gut microbiome, the skin microbiome, the oral microbiome, or the lung microbiome, among others. Thus, in addition to the application of insect repellency, as discussed herein, embodiments of the general platform described herein may also be applicable in a wide range of applications, including gut health, atopic dermatitis, body odor, oral health, and the like. In any of the embodiments described herein the methods may be useful for modulating a microbiome of a subject, wherein the subject is a human or a domesticated animal.

### Oral Health

The composition of the oral microbiome differs from one intraoral site to another, reflecting in part the host response and immune capacity at each site. By focusing on two major oral infections, periodontal disease and caries, new principles of disease emerge. Periodontal disease affects the soft tissues and bone that support the teeth. Caries is a unique infection of the dental hard tissues. The initiation of both diseases is marked by an increase in the complexity of the microbiome. In periodontitis, pathobionts and keystone pathogens such as Porphyromonas gingivalis appear in greater proportion than in health. As a keystone pathogen, P. gingivalis impairs host immune responses and appears necessary but not sufficient to cause periodontitis. Historically, dental caries had been causally linked to Streptococcus mutans. Contemporary microbiome studies now indicate that singular pathogens are not obvious in either caries or periodontitis. Both diseases appear to result from a perturbation among relatively minor constituents in local microbial communities resulting in dysbiosis. Emergent consortia of the minor members of the respective microbiomes act synergistically to stress the ability of the host to respond and protect. In periodontal disease, host protection first occurs at the level of innate gingival epithelial immunity. Secretory IgA antibody and other salivary antimicrobial systems also act against periodontopathic and cariogenic consortia. When the gingival immune response is impaired, periodontal tissue pathology results when matrix metalloproteinases are released from neutrophils and T cells mediate alveolar bone loss. In caries, several species are acidogenic and aciduric and appear to work synergistically to promote demineralization of the enamel and dentin. Whereas technically possible, particularly for caries, vaccines are unlikely to be commercialized in the near future because of the low morbidity of caries and periodontitis.

Periodontal disease is the most common infectious disease affecting tooth-supporting structures. Left untreated, periodontitis can lead to, or aggravate existing systemic conditions such as cardiovascular disease, diabetes, pulmonary diseases, and obesity. In dentistry, understanding the changes in the oral microbiome that foretell the early stages of periodontitis and dental caries, the most prevalent chronic oral diseases, may allow the better diagnosis and treatment before the appearance of the telltale clinical manifestations of these diseases (such as tissue damage in periodontal pockets or dental hard tissue loss). The emergence and evolution of antibiotic resistance in periodontal pathogens has affected the therapeutic success rates for this disease.

The diseased microbiome is enriched in metabolic functions that are consistent with a parasitic lifestyle made possible by the availability of nutrients derived from the degradation of host tissue and from bacterial cells destroyed by the host immune response. Among the host immune response are functions for fatty acid metabolism and acetyl-coenzyme A degradation, aromatic amino acid degradation, ferrodoxin oxidation, and energy-coupling factor (ECF) class transporters. The periodontal pocket has been previously shown to be enriched for such nutrients in patients with periodontitis (çiçek et al., 2005). Several of these metabolic functions have also been associated with an intracellular lifestyle (e.g. fatty acid metabolism), or with anaerobic metabolism (e.g. ferrodoxin oxidation, and acetyl-CoA degradation), highlighting the diversity of survival strategies employed by the microbes inhabiting the periodontal pocket during disease. Also enriched in disease are a number of virulence factors such as the presence of conjugative transposons, type IV secretion systems, and the biosynthesis of toxic factors (e.g. acetone, butanol, and ethanol biosynthesis), as well as the Lipid-A of lipopolysaccharide (LPS) biosynthesis. LPS is a group of molecules known to trigger host immune response and inflammation and their enrichment in disease provides a possible explanation for the systemic impact of periodontitis on the human host.

Finally, the periodontal disease samples are enriched in a number of functions related to drug and metal resistance (mercury, cobalt-zinc-cadmium). Mercury resistance has been previously characterized as a common feature of oral bacteria, even in the absence of mercury-containing amalgam, and is frequently associated with antibiotic resistance. The role drug resistance plays in disease is, however, unclear as antibiotic resistance factors are present in both healthy and diseased samples. Orally healthy samples include at the species-level phylotypes, Corynebacterium durum, Corynebacterium matruchotii, unclassified Neisseria and Streptococcus.

Comparatively, only a few pathways are significantly enriched in the healthy microbiome (or depleted in the diseased microbiome), including pathways for fatty acid biosynthesis, purine metabolism, and glycerol-3-phosphate metabolism. Certain fatty acids have been shown to have a protective role in periodontal health (Campan et al., 1997; Kesavalu et al., 2007) and it is possible that some of these are synthesized by the healthy microbiota. However, most of what is known about the role of fatty acids in periodontal health is based on nutritional studies and the contribution of the oral microbiota has yet to be characterized. Glycerol-3-phosphate is a lipid metabolite that has been shown to occur in higher concentration in periodontal disease samples (Barnes et al., 2009). Our study hints that a possible explanation for this observation is a decrease in the ability of the disease microbiome to metabolize this compound. Also enriched are genes related to homoserine metabolism, possibly related to quorum sensing functions within the healthy microbiome, as homoserine lactones are frequently used as quorum sensing molecules in oral bacteria. The enrichment, within healthy samples, of the reactions downstream of homo-serine lactone pathway may indicate a fully functioning quorum sensing system, allowing for the communication between organisms that is the hallmark of a healthy biofilm system. In poly-microbial biofilms it has been shown that mutants lacking quorum-sensing molecules, while able to construct biofilms, are unable to obtain the correct structure and thickness. The depletion of pathways related to quorum sensing in diseased samples may indicate a possible cause of disease progression due to the inability of the healthy microbiome to maintain a protective biofilm. (Liu et al., 2012).

Periodontitis is a kind of infectious disease initiated by the colonization of subgingival periodontal pathogens, which cause destruction of the ligament and alveolar bone supporting the teeth and ultimately, results in the loss of the affected teeth and with the resultant loss of quality of life. According to the latest official classification system for periodontal diseases from the American Academy of Periodontology, periodontitis can be classified into two main types: chronic and aggressive. Chronic periodontitis (ChP) is a slowly progressive disease, most prevalent in adults and usually associated with marked accumulation of biofilm and calculus. Conversely, aggressive periodontitis (AgP) belongs to a group of rare periodontal diseases initiated at a young age with rapid attachment loss, which is not necessarily correlated with high levels of biofilm and calculus.

The microorganisms in a dental biofilm are believed to be involved in the pathogenesis of periodontitis; in particular, subgingival bacteria plays an important role in its initiation and progression. Decades of investigations have tried to identify a microbiological element of AgP to help in the differential diagnosis from ChP, however, the notion that AgP has a distinct microbiological pathogenesis from ChP has still not been confirmed.

The progress of understanding oral microbiology is dependent on the development of microbial research techniques. The emergence of the next-generation sequencing (NGS) of bacterial 16S ribosomal RNA (rRNA) gene makes it possible to show a nearly unbiased view of the bacterial composition, which has the advantage of detecting non-culturable bacteria, fastidious bacteria, even novel microbes. In recent years, NGS has been widely used to analyze subgingival bacterial composition and to characterize compositional shifts between periodontal health and disease (Liu et al., 2012; Abusleme et al., 2013; Li et al., 2015; Park et al., 2015; Han et al., 2017). Some studies observed the striking differences between the microbiota of healthy sites (probing depth≤3 mm) and diseased sites in ChP subjects (Ge et al., 2013; Pérez-Chaparro et al., 2018), as it is known that the composition of the subgingival microbiome varies according to probing depths possibly because of dissimilar ecological parameters such as oxygen tension (Loesche et al., 1983; Abusleme et al., 2013). However, there is scarce evidence on the shift of subgingival microbiome in individual tooth sites of both types of periodontitis during disease development. (Shi et al., 2018).

Oral biofilms provide physical protection from dietary acid and together with bacterial metabolic acids cause the resting pH of the biofilm to fall below neutral. This is then followed by the re-establishment of a neutral environment by chemical interactions mediated by the saliva within the biofilm. Such pH fluctuations are often responsible for the cyclic demineralization, then remineralization of teeth, a process necessary for tooth maturation. However, since the advent of farming and especially since the industrial revolution, the increase in consumption of carbohydrates, refined sugars and acidic drinks has changed the ecology of biofilms. Biofilm biodiversity is significantly reduced together with a proliferation of acidogenic and aciduric organisms, tipping the balance of the mineralization cycle towards net mineral loss and hence caries. In addition, the consumption of acidic drinks in today's societies has removed the protective nature of the biofilm, leading to erosion. Erosion and caries are modern-day diseases and reflect an imbalance within the oral biofilm resulting in the demineralization of teeth (Kaidonis and Townsend, 2016).

In caries, a number of metabolic pathways contribute to susceptibility and causation, including: the central carbon metabolism, the Embden-Meyerhof-Parnas (EMP) pathway, the pentose-phosphate pathway, and the tricarboxylic cycle in supragingival biofilm and specific oral bacteria, S. mutans, Streptococcus sanguinis, Actinomyces oris, and Actinomyces naeslundii.

The microbiome of the gingival cleft is of great interest in human dentistry because the two most important diseases of the teeth and periodontium in human patients, dental caries and periodontitis, are related to changes in the relative contribution of various potentially pathogenic bacteria in the complex biofilm referred to as dental plaque (Wade, 2013). As a result, there is considerable information on the oral microbiome of human patients, how this is associated with different disease conditions, and how this is influenced by diet. This includes studies using ancient deoxyribonucleic acid (DNA) which have shown that systematic changes in the microbiome are correlated with changes in diet, in both a contemporary and an evolutionary sense.

Cats, like humans, are very commonly affected by periodontal disease, with a consensus that it is the most common disease of feline patients in developed nations (O'Neill et al., 2014). While periodontal disease is seen in cats of all ages, it is generally considered to progress with age, although its extent and severity are impacted on by such factors as diet and co-morbid disease (especially kidney disease and infection with feline immunodeficiency virus or feline calicivirus). Indeed, some feline diets are specifically formulated to prevent or ameliorate the severity of feline periodontal disease. Although cats do not get dental caries, they are commonly afflicted by resorptive lesions (RLs), the origins of which are poorly understood but are characterized by erosion of enamel, dentin or cementum. Feline skulls analyzed in retrospective studies of museum and zoo specimens demonstrate a low prevalence of RLs before the 1960s, which may suggest causal relationships with altered husbandry of domesticated cats including feeding practices.

The third disease condition of the feline oral cavity is referred to as feline chronic gingivostomatitis (FCGS). Although there is strong evidence to support the involvement of feline calicivirus (FCV) in some cases, the inability to recreate the disease in a naïve population and the success of treatments such as full-mouth dental extractions in many cases have cast doubts on a singular role for FCV and raised suggestions that this disease may be influenced by the nature of the host's response and derangements (dysbiosis) of the oral microbiological flora.

The microbiome of the gingival cleft impacts additionally on common and important feline disease conditions outside the oral cavity. Infections resulting from cat bites, in both feline and human patient, are typically polymicrobial with a preponderance of obligate anaerobes and facultative anaerobic bacteria, of which only some are cultivatable using routine laboratory methods. Likewise, infections of the upper and lower respiratory tract and pleura of cats often involve oropharyngeal flora, including facultative and obligate anaerobic bacteria. Thus, chronic sinonasal cavity disease, pneumonia and especially purulent pleurisy (pyothorax) can involve cultivatable and likely uncultivable anaerobic bacteria, as well as facultative anaerobic bacteria such as Pasteurella spp.

Early studies on cultivatable organisms within the feline oral cavity found shifts towards a higher proportion of anaerobic gram-negative rods in cats with higher gingival index scores, with prominence of bacteria within Bacteriodetes such as Porphyromonas sp. possessing suitable virulence factors capable of causing periodontal disease, and with these virulence factors inciting an appropriate humoral immune response. To date, there have only been a handful of in-depth genetic studies of the feline oral microbiome, and these have not considered the contribution of diet to the observed findings. (Adler et al., 2016).

Like humans, pets and animals have a proclivity to caries lesions and periodontis. Large proportions of the cultivable microbiota from canine plaque were Actinomyces species (12%) and Corynebacterium species (5%). These genera have recently been implicated in canine periodontitis (Takada and Hirasawa, 2000), and it has been suggested that they may play the same role in canine periodontitis that P. gingivalis plays in human periodontitis. This suggestion is based upon their findings that the proportion of these genera possessing a trypsin-like activity (TLA) is increased in periodontitis sites compared with the proportion in healthy sites and may explain in part the absence of P. gingivalis from plaque collected in this study if they are able to compete for a similar niche.

Accordingly, some embodiments provided herein relate to methods and systems for modulating the oral microbiome. In some embodiments, the methods and systems modulate the oral microbiome of a subject by enhancing the oral microbiome biochemical pathways to improve oral health, including to prevent, ameliorate, or otherwise inhibit periodontal disease, including caries. In some embodiments, the methods include determining compounds being consumed and compounds being accumulated in an oral microbiome of a subject suffering from a dysregulation in oral health, in comparison to compounds from an oral microbiome of a healthy subject, and modulating the oral microbiome of the subject suffering from dysregulation in oral health to improve oral health. In any of the embodiments provided herein, the subject is a human or an animal, such as a domesticated animal.

### Body Odor

Human axillary odor is commonly attributed to the bacterial degradation of precursors in sweat secretions. Volatile organic compounds (VOCs) emanating from this area give rise to the canonical axillary odor (commonly referred to as “body odor”).

Age, sex, genetic factors, environmental factors (climate or stress situation), hygiene and the use of cosmetics may contribute to body odor by influencing the quantity and quality of secretions, or the types of bacteria present on skin. A culture-based approach used previously to isolate odor-generating bacteria was successful in identifying Corynebacterium and Staphylococcus species, in particular, Corynebacterium striatum, C. jeikeium and Staphylococcus haemolyticus as implicated in the generation of odorous volatiles.

Two distinct types of axillary microbiota dominated by either coryneforms or cocci have been reported, the former being more prevalent in males and contributing to a more pronounced body odor (Taylor et al., 2003). Similarly, under conditions of high nutrient supply and humidity, the growth of coryneforms is favored, and they may also be able to suppress the growth of cocci (Jackman and Noble, 1983). Women have 75% more apocrine glands in their armpits than men, but male apocrine glands are larger and may be more active in order to supply nutrients for bacterial growth.

Human body odor is a complex mixture of VOCs. Organoleptic and analytical chemistry methodologies have revealed that axillary odor derives from a complex mixture of C₂-C₁₁ normal, branched, and unsaturated acids, with the main components being (E)-3-methyl-2-hexenoic acid (E-3M2H) (Pierce et al. 1995; Zeng et al. 1991, 1992, 1996a, b) and 3-hydroxy-3-methylhexanoic acid (3H3M) (Natsch et al. 2003), as well as volatile sulfur compounds, particularly (S)-3-methyl-3-sulfanylhexan-1-ol) (Hasegawa et al. 2004; Natsch et al. 2004; Troccaz et al. 2004). The latter sulfur-containing compounds often are present at very low levels but have high odor impact (i.e., low olfactory threshold). Volatile steroids, such as 5α-androst-16-en-3-one (androstenone) and 5α-androst-16-en-3β-ol (androstenol), originally were thought to play a role in axillary odor (Bird and Gower 1981), but later were found to be minor sensory and analytical contributors relative to the organic acids (Zeng et al. 1996a, b). Apocrine secretions, which contain the precursors to axillary odor, are odorless upon secretion and become odorous after interaction with the axillary microbial communities residing on the skin's surface.

Hygiene habits such as shaving axillae or use of cosmetics and antiperspirants (APs) may alter the odor profile by changing sweat volumes, the microbiota profile and its metabolic activity. Some cosmetics may contain nutrients such as glycerine, amino acids and hydrolyzed collagen for the resident microbiota, or they may contain antimicrobials that increase the presence of resistant strains on skin. S. hominis OTUs were positively associated with odors. Our observation that the genus Corynebacterium correlates with body odors is also in agreement with the data from a culture-based study. The predominant colony type of aerobic axillary corynebacteria had the best sequence match to C. tuberculostearicum. Other corynebacteria such as C. striatum and C. jeikeium have been reported as odor-generating microorganisms in the underarms. Our data indicate that the most abundant OTU from the genus Corynebacterium is assigned to C. tuberculostearicum. The axillae support a dense bacterial population dominated by two types, Staphylococcus and Corynebacterium. A strong correlation has been found between a dense population of corynebacteria and robust axillary odor production. However, subtle differences in the amounts of short, C₂-C₆, straight- and branched-chain organic acids. Higher amounts of all straight-chain acids: acetic, propanoic, butyric, valeric, and hexanoic; however, when branch-chained acids (isobutyric, 2-methylbutyric, and isovaleric acid) were examined. Longer chain odorants 3-Methyl-2-hexenoic acid, 2-octenoic acid, and 7-octenoic acid are associated with body odor, while lower levels of malodorant precursors such as N-acyl glutamine are found in unaffected individuals. The branched-chain volatile acids, isovaleric acid and 2-methylbutyric acid, stem from the metabolism of leucine and isoleucine are found in high levels, respectively in body odor.

Accordingly, some embodiments provided herein relate to methods and systems for modulating a microbiome for improving, enhancing, or eliminating body odor. In some embodiments, the methods and systems modulate a microbiome, such as a skin microbiome of a subject by enhancing the microbiome biochemical pathways to generate compounds for improving, enhancing, or eliminating body odor. In some embodiments, the methods include determining compounds being consumed and compounds being accumulated in a microbiome of a subject suffering from poor body odor, in comparison to compounds from a microbiome of a subject having good or no body odor, and modulating the microbiome of the subject suffering from poor body odor to improve body odor. In any of the embodiments provided herein, the subject is a human or an animal, such as a domesticated animal.

### Atopic Dermatitis

Atopic dermatitis (AD) is a common chronic inflammatory skin disease affecting ˜10-20% of the general population. AD is characterized by disturbances in epidermal barrier function and hyperactive immune response. Recently, changes in the skin and intestinal microbiome have been analyzed in more detail. The available data suggest a link between disturbed skin microbiome and course of the disease. Flares of the disease are associated with an expansion of Staphylococcus aureus on lesional skin and a substantial loss of biodiversity in skin microbiome. Staphylococci exoproteins and superantigens evoke inflammatory reactions in the host. Skin microbiome includes superficial stratum corneum that is affected by environmental factors such as exposure to germs and cleansing. Available evidence argues for a link between epidermal barrier impairment and disturbances in skin microbiome in AD.

Resident skin bacteria are influenced by topological and endogenous factors of skin and can be modulated by external factors such as clothing, hygiene, topical treatments and skin care products. There are gender differences in skin microbiome as well. Skin microbiomes differ between children and adults (described in the following). Bacteria are not uniformly distributed in skin. There is a superficial and a deeper compartment in the human stratum corneum. After injury, a neo-microbiome is produced from the deeper compartment, which can be regarded as the indigenous microbiome. Furthermore, bacteria are consistently detectable also in deeper skin layers such as the dermis and the subcutaneous adipose tissue. A balanced resident skin flora is a protective measure.

Among the Gram positive Staphylococcus species, Staphylococcus epidermidis is the dominant type in healthy skin with the ability to inhibit the growth of Staphylococcus aureus. In children, colonization of skin by S. epidermidis and S. cohnii during the first year of life has a protective effect on the development of AD. Disturbances in cutaneous microbiome represent an independent risk factor for the development of AD. In ˜90% of patients suffering from AD, the skin becomes colonized by S. aureus of which 50% are toxin producing. These toxins can contribute to inflammation and skin barrier dysfunction via activating the host inflammasomes. High iron and low ascorbic acid concentrations are found in the dermis of atopic dermatitis patients.

Healthy individuals are colonized with a diverse microbiota, with significant interpersonal variabilities, and differences across body sites in the same individual (Grice and Segre, 2011). Canine skin is no different, and it is colonized with an even more diverse microbiota, also with significant differences across individuals and body sites (Rodrigues Hoffmann et al., 2014). The microbiota diversity is reduced in both people and dogs with atopic dermatitis (AD). In addition, the role of beneficial bacteria, such as coagulase negative Staphylococci, has been largely demonstrated (Gallo, 2015). Furthermore, it is clear that the presence of pathogenic bacteria, such as Staphylococcus aureus, in people and S. pseudintermedius in dogs, may induce exacerbation/reacutization of the clinical signs of AD in people and dogs, respectively (Kong et al., 2012, Santoro et al., 2015, Williams and Gallo, 2015). Furthermore, dogs are naturally affected by AD, they show clinical and immunological similarities with the human disease, and they share much of the same environment with their owners (Santoro and Marsella, 2014). Thus, dogs with AD represent a perfect model to study host-microbiome interaction, mirroring the human AD.

Accordingly, some embodiments provided herein relate to methods and systems for modulating the skin microbiome. In some embodiments, the methods and systems modulate the skin microbiome of a subject by enhancing the skin microbiome biochemical pathways to improve skin health, including to prevent, ameliorate, or otherwise inhibit a skin disorder, such as atopic dermatitis. In some embodiments, the methods include determining compounds being consumed and compounds being accumulated in a skin microbiome of a subject suffering from a skin disorder, such as atopic dermatitis, in comparison to compounds from a skin microbiome of a healthy subject, and modulating the skin microbiome of the subject suffering from a skin disorder to improve skin health, including to improve symptoms of atopic dermatitis, or to prevent, inhibit, or ameliorate atopic dermatitis. In any of the embodiments provided herein, the subject is a human or an animal, such as a domesticated animal.

### Gut Health

The main microbiome bacteria found among humans is associated with the digestive track, and may weigh an estimated five pounds. Microbes have a vested interest in maintaining a symbiosis with their host, as a healthy host means that their environmental niche is maintained for their own successful growth and prosperity of a species. As residents of the gut, the microbiome is involved in many metabolic processes, from the breakdown of food, to short fatty acid synthesis, and even the production of vitamins. Vitamins are critical micronutrients that are required for coenzymes and some, such as Vitamin B12, are not produced by the host. Vitamins produced by microbes are predominantly absorbed by the host in the colon, where it is known that thiamine, folates, biotin, riboflavin, pantothenic acid, and menaquinones can be absorbed. Providing the host with these beneficial micronutrients can help maintain dietary host homeostasis as well as boost immune system function (Engevik et al., 2017, and Leblanc et al., 2017). In fact, the microbiome has been shown to produce B vitamins like thiamine, B12, and riboflavin. In addition to these, some species are involved in making Vitamin K. The potential to encourage the microbiome to make vitamins more readily available to the host is of great interest considering that modern society has a greater propensity to process foods more and lose important vitamins and minerals along the way (Rowland et al., 2018).

Much in the same way that the gut microbiome is involved in the production of vitamins in humans, the microbiomes of various pets and animals are also suggested to have similar benefits to their hosts. For example, in a comparative analysis between species, a group looked at the similarities between the microbiomes of mice, pigs, and dogs with that of humans and found that dogs actually share a microbiome similarity of more than 60% with their human counterparts (Coelho et al., 2018). This would suggest that many of these gut species are in fact performing the same type of metabolic processes involved in digestive health and host nutritional uptake.

Gut commensals need to make-do with whatever the host is consuming in their diet in order to be successful. In many situations, however, the diet does not provide all of the elements required for protein production, which is an essential part that provides the internal machinery that maintains homeostasis. In the case of amino acids, environmental bacteria more often than not need to supplement what they find in the environment during digestion with amino acids that they must make on their own. It has been shown that bacterial species will make certain amino acids de novo so that they can grow and thrive in the digestive system. Contrary to microbes, humans cannot make all of the amino acids that are required for protein production, and so they must obtain them as part of their diet. Luckily, their own microbial residents often times need to produce these same essential amino acids that may not be present in the diet. As bacterial growth and death is a natural process in the gut, the manufactured amino acids will then be made available to the host for amino acid absorption and can therefore fulfill at least part of the host's nutritional requirements (Metges et al., 1999).

Bacteria in animal guts must produce certain amino acids much like those of the human gut when they are not available in the animal's diet. De novo synthesis of amino acids and subsequent uptake by the host animal has been demonstrated in pigs (Torrallardona et al., 2003). This same uptake process of pigs most likely occurs in other mammals and pets alike, given the need for proper nutrition from whatever dietary source an animal is provided. This idea is also supported by the similarity between the microbiomes of humans and dogs (Coelho et al., 2018).

Accordingly, some embodiments provided herein relate to methods and systems for modulating the gut microbiome. In some embodiments, the methods and systems modulate the gut microbiome of a subject by enhancing the gut microbiome biochemical pathways to improve gut health, including to improve gut metabolism, boost immune function, or improve digestive health and nutritional uptake. In some embodiments, the methods include determining compounds being consumed and compounds being accumulated in a gut microbiome of a subject suffering from poor gut health, in comparison to compounds from a gut microbiome of a subject experiencing good gut health, and modulating the gut microbiome of the subject suffering from poor gut health to improve gut health, including to improve gut metabolism, boost immune function, or improve digestive health and nutritional uptake. In any of the embodiments provided herein, the subject is a human or an animal, such as a domesticated animal.

### Fragrances

Bacteria have long been used to produce various industrial and medical products. Some bacteria of the microbiome, however, naturally have the ability to produce compounds that are commonly known to be associated with fragrances that can have cosmetic application. For example, 2-phenylethanol is a fragrance that is responsible for the rose like smell that we associate with flower bouquets (Sakai et al., 2007). Exploitation of these natural pathways that have been outlined in the KEGG database can result in better, long-lasting products for the cosmetic industry (See enzyme 1.1.1.90, that results in 2-Phenylethylalcohol).

Accordingly, some embodiments provided herein relate to methods and systems for modulating a microbiome to produce a fragrance. In some embodiments, the methods and systems modulate a microbiome to produce a fragrance that is desirable, and which may be formulated in a product for application to improve fragrance. For example, a formulation may be a cosmetic or therapeutic formulation, where it is desirable to improve fragrance of the formulation or to enhance a fragrance upon application of the formulation to a subject.

### Newborn Immunity

The specific mechanisms that lead to the formation of the human milk microbiota are still unknown; however, there are different hypothesis that can explain the origin of milk-associated bacteria. Indeed, some microorganisms belonging to the maternal skin or infant's oral cavity may become an integral component of the milk microbiota by means of a milk flow back into mammary ducts during lactation (Rodriguez, 2014). This mechanism may justify the presence of cutaneous and oral bacteria that are recovered in the milk microbiota, such as Streptococcus spp. and Staphylococcus spp. (Gao et al., 2007; Grice et al., 2009). Interestingly, human milk contained also a great number of intestinal bacteria, which may spread from the maternal intestinal environment by a mechanism involving dendritic cells (DCs) and CD18⁺ cells (Rodriguez, 2014); these cellular types would be able to capture intestinal microorganisms from the gut lumen and transfer them to lactating mammary glands by means of translocation, which results to be increased during late pregnancy and lactation (Rodriguez, 2014). Consequently, the milk microbiota can shape the initial intestinal microbiome of newborns, together with the maternal intestinal and vaginal microorganisms that are ingested by the neonate during the passage through the birth canal (Houghteling and Walker, 2015). Human milk can stimulate the proliferation of numerous Bifidobacterium and Lactobacillus strains, the main probiotic microorganisms present in the gut, creating an acidic environmental rich in short chain fatty acids (SCFAs) with a protective and nutritive role at intestinal level (Bode, 2012; Walker and Iyengar, 2015). The constant intake, during lactation, of bacteria contained in the human milk leads to the formation of a transient intestinal microbiota that deeply impacts on the newborn's development, acting mainly on the maturation of his immune system (Houghteling and Walker, 2015). Indeed, several studies underscored the strict link between the gut microbiota signals, the mucosal host defense and the maturation of immune system, both at intestinal and systemic level (Smith et al., 2007; Sekirov et al., 2008; Walker and Iyengar, 2015). It has been shown that an altered colonization of newborns' gut may lead to a persistent intestinal dysbiosis and consequently, to immune-mediated and metabolic diseases during infancy and childhood (Gareau et al., 2010; Johnson and Versalovic, 2012). Moreover, breast-fed newborns have shown to possess a more stable intestinal bacterial population and a well-balanced mucosal immune response if compared to the formula-fed ones (Gronlund et al., 2000; Bezirtzoglou et al., 2011); indeed, a healthy intestinal microbiota can induce specific T cell responses and modulate substrates oxidation, decreasing the impact of autoimmune and allergic diseases not only during childhood but also in adulthood (Guaraldi and Salvatori, 2012; Palma et al., 2012). Finally, breastfeeding has been observed to have a protective role against respiratory and gastrointestinal infections between the ages of 7 and 12 months, leading to a general improvement of symptoms associated to gastrointestinal infections (Duijts et al., 2010).

Intestinal bacteria can also stimulate lymphoid elements and positively influence the maturation of both innate and adaptive immune system, as clearly demonstrated by studying germ-free animals (Cash and Hooper, 2005). It has been shown that in germ-free mice the villus capillaries develop poorly during weaning and remained in this condition also during adulthood, suggesting that the intestinal microbiota is fundamental for intestinal blood vessel to be completely developed (Martin et al., 2010). More interestingly, intestinal bacteria can promote B cell development in Peyer's Patches and increase the production of mucosal IgA, the main antibody class in secretions that acts as first line of defense (Martin et al., 2010).

Moreover, bacterial surface-expressed or secreted ligands can interact with specific receptors on mucosal immune system and enterocytes leading to a self-limited inflammatory response for preventing pathogen mucosal penetration (Round and Mazmanian, 2009; Walker and Iyengar, 2015).

As underlined by Latuga et al. (2014), all newborns have an immature immune system and the cord blood rich in anti-inflammatory T regulatory cells; furthermore, infants have a high T helper 2 (Th2) that promotes humoral immunity with the production of IL4, IL6 and IL21, thus promoting an increased B cell response and potentially, a higher allergic sensitization (Latuga et al., 2014). The pivotal role of milk-associated microbiota in influencing the neonates' immune system is over-emphasized by the cytotoxic function promoted by microbial ligands in breast milk (Donnet-Hughes, 2008). Indeed, in vitro stimulation of DCs with lipopolysaccharide can lead to T-cells differentiation, supporting the hypothesis that mature milk may implement the maturation of cytotoxic Th1 cells and improve their activity against infections (M′Rabet et al., 2008). Probably, commensal colonic bacteria may stimulate the release of specific cytokines that create a balanced microenvironment suitable for naive Th0 cells to ripen toward Th1 cell type (Walker and Iyengar, 2015).

Bacteroides is a bacterial genus that is very abundant in human colostrum and it may have a main role in the early stages of newborns' gut colonization, as reported by Mazmanian and Kasper (2006). In particular, the polysaccharide A located on the surface of Bacteroides fragilis can interact with Toll receptor 2 on intestinal DCs to stimulate cytokine production which, in turn, favor the proliferation of FOXP3 T cells in the lamina propria. FOXP3 belongs to the forkhead transcription factor family bindweed in the expansion of regulatory T cells, thus having a suppressive role in the host's immune system (Kim, 2009). Therefore, it is clear that a correct stimulation of neonates' intestinal environment is fundamental for the physiological development of mucosal immune system and tolerance; the latter, in particular, is extremely important to avoid developing allergy or autoimmune diseases (Walker and Iyengar, 2015). Indeed, germ-free animals cannot develop tolerance due to the lack of intestinal bacteria and only the adequate colonization of newborns' gut can lead to a complete tolerance generation (Karlsson et al., 1999; Olszak et al., 2012).

Consequently, breastfeeding is essential for oral tolerance in newborns, as it is extremely important for the establishment of local and systemic immune tolerance to antigens ingested during lactation (Verhasselt, 2010). Infants are daily exposed to specific antigens, part of which belong directly to the human milk microbiota, and that can translocate across the intestinal barrier, being involved in the presentation by antigen-presenting cells to T lymphocytes. Furthermore, bacteria located in the human milk are fundamental to correctly stimulate the Peyer's patches, increasing the number of IgA-producing plasma cells in the intestinal environment of newborns (Gross, 2007). Consequently, IgA can trap food antigens favoring their elimination by specific enzymes, avoid the adherence of viruses and microorganisms to intestinal mucosa also counteracting the proliferation of pathogens and exert a direct immunomodulatory activity (Verhasselt, 2010).

Finally, the oral tolerance seems to be actively involved in the prevention of allergic diseases onset in babies, avoiding also the impact of respiratory and gastrointestinal infections during the early stages of their life (Lack, 2008).

Accordingly, some embodiments provided herein relate to methods and systems for modulating the milk microbiome. In some embodiments, the methods and systems modulate the milk microbiome of a subject by enhancing the milk microbiome biochemical pathways in order to improve health of a nursing offspring, including to improve immunity, gut health, oral health, or general health of the offspring. In any of the embodiments provided herein, the subject is a human or an animal, such as a domesticated animal.

### Chronic Obstructive Pulmonary Disease

Lung cancer (LC) is the main cause of overall cancer mortality. Even with joint efforts for earlier detection and potent chemotherapy, the overall 5-year survival rate remains disappointing. Chronic obstructive pulmonary disease (COPD) has become the fourth leading cause of death in the world, with identification as an independent LC risk factor. Moreover, COPD and LC have shared etiology, such as aging, environmental and occupational exposure, inflammation and oxidation, and epigenetics changes. The development of COPD and LC in ever smokers (i.e., current and former smokers) likely requires decades of repetitive exposure of the airway to cigarette smoke. Lung carcinogenesis consists of a cascade of key etiological changes prior to clinical cancer diagnosis, with some of them being identified as causal events in COPD genesis. Focusing on primary prevention by targeting these biological changes may thus be of high value for reducing the overall mortality in smokers. Moreover, chemoprevention strategies developed for one may help for the other, and interventions targeting on these communal pathogeneses may yield great success for the prevention of both.

The lung was thought to be a sterile organ until the first report identifying a lung microbiome in healthy subjects. Since then, numerous studies have explored the diverse microbiota in the human lung by using molecular techniques and have found evidence that lung microbiomes might change in COPD pathogenesis. COPD is characterized by small airway inflammation, which intensifies with pathogenesis. Gammaproteobacteria, a typical lung microbiome class which are represented by Pseudomonas aeruginosa, were found grow rapidly under chronic inflammatory conditions and increase in lungs during disease. Studies probing into the relationships between the lung microbiome and inflammatory response found there might be a feedback loop: gammaproteobacteria feed on inflammatory products while encoding components to promote inflammation. Several studies have demonstrated accelerated lung function decline with recurrent lower respiratory tract infections, together with the exacerbation of lung function decline by bacteria. Lung microbiomes may thus be of great importance in the pathogenesis of lung function impairment and COPD. Nutrition can be promising for the prevention of lung function decline caused by possible imbalance of the lung microbiome. Since several nutrients possess anti-inflammatory effects, supplementation of them might be helpful for improving lung function by breaking the feedback loop between the abnormal lung microbiome and inflammatory response. Lung microbiome composition was found change with dietary vitamin D, and serum 25-hydroxyvitamin D (25(OH)D) levels were inversely associated with Pseudomonas in the lung of animal models. However, for now, limited studies have investigated the benefit effect of nutrients on lung microbiome balance, due to the lack of more accurate knowledge about the lung microbiome and its role in the pathogenesis of lung disease.

Accordingly, some embodiments provided herein relate to methods and systems for modulating the lung microbiome. In some embodiments, the methods and systems modulate the lung microbiome of a subject by enhancing the lung microbiome biochemical pathways to improve lung health, including to prevent, inhibit, or ameliorate lung inflammation or oxidation, or COPD. In some embodiments, the methods include determining compounds being consumed and compounds being accumulated in a lung microbiome of a subject suffering from poor lung health, in comparison to compounds from a lung microbiome of a subject experiencing good lung health, and modulating the lung microbiome of the subject suffering from poor lung health to improve lung health. In any of the embodiments provided herein, the subject is a human or an animal, such as a domesticated animal.

### Compositions

Some embodiments provided herein relate to compositions that modulate a microbiome biochemical pathway. In some embodiments, the compositions modulate a skin, gut, oral, lung, or other microbiome, or any combination thereof. In some embodiments, the composition modulates a microbiome biochemical pathway to generate a compound that confers one or more desirable effects to the subject, including, for example, a compound that confers insect repellency, a compound that prevents, inhibits, treats, or ameliorates a disorder, such as an oral disorder, a lung disorder, a skin disorder, a gut disorder, a compound to promotes health, a compound that generates desirable odors or fragrances, or a compound that eliminates or reduces undesirable odors.

The compositions may be formulated for any suitable route of administration, depending upon the microbiome that is to be modulated. Thus, for example, suitable routes of administration may include topical, parenteral, oral, intraocular, or by inhalation.

In some embodiments, the composition is formulated for topical application, for example to modulate a skin microbiome. Topical formulation may include, for example, a spray, an aerosol, a powder, a foam, a foamable liquid, a gel, a serum, a spritz, a lotion, a cream, a sunscreen, an ointment, an oil, a solution, a vapor, an emollient, a paste, or a salve.

In some embodiments, the composition is formulated for oral ingestion by a subject, for example for modulating a gut or oral microbiome. In some embodiments, the formulation is formulated as a tablet, pill, capsule, granule, gummy, dragee, liquid, gel, syrup, slurry, spray, or suspension. In some embodiments, the composition is in the form of a tablet, a film coated tablet, a gel cap, a caplet, a pellet, or a bead.

In some embodiments, the composition is formulated for administration to airways. In some embodiments, the composition is formulated as a drop, a spray, an aerosol, a vapor, a nebulized compound, or an inhalant for administration through the airways and to regions associated with the airway, such as the nasal cavity, the oral cavity, or the lungs. In some embodiments, a compound of Formula (I) is formulated for intranasal administration alone or in combination with an additional therapy described herein, including one or more of an androgen deprivation therapies, anti-estrogen therapies, biologic therapies, virus-based therapies, surgeries, chemotherapies, such as taxane-based chemotherapy agents or platinum-based antineoplastic agents, radiation therapies, statin therapies, repurposed drug therapies, small molecule inhibitor therapies, therapeutic antibody therapies, or immunotherapies, or any combinations thereof and one or more appropriate pharmaceutically acceptable carriers or excipients for intranasal administration.

In any of the embodiments, the formulation may include acceptable carriers, excipients, diluents, stabilizers, emollients, binders, or buffers suitable for the specific route of administration.

An acceptable carrier refers to a substance, not itself a therapeutic agent, which may facilitate the incorporation of a compound into cells or tissues. The carrier may be a liquid for the dissolution of a compound to be administered by ingestion. The carrier may be a vehicle for delivery of a therapeutic agent to a subject. The carrier may improve the stability, handling, or storage properties of a therapeutic agent. The carrier may facilitate formation of a dose unit of a composition into a discrete article such as a capsule, tablet, film coated tablet, caplet, gel cap, pill pellet, or bead, and the like suitable for administration to a subject.

A diluent refers to an ingredient in a pharmaceutical composition that lacks pharmacological activity but may be necessary or desirable. For example, a diluent may be used to increase the bulk of a potent drug whose mass is too small for manufacture or administration. It may also be a liquid for the dissolution of a drug to be administered by injection, ingestion or inhalation. A common form of diluent in the art is a buffered aqueous solution such as, without limitation, phosphate buffered saline that is physiologically compatible with human cells and tissues.

An excipient refers to an inert substance that is added to a pharmaceutical composition to provide, without limitation, bulk, consistency, stability, binding ability, lubrication, or disintegrating ability etc., to the composition. A diluent is a type of excipient.

The compositions described herein may be prepared by a process as described herein. Thus, in some embodiments, a composition for modulating a microbiome in a subject includes a compound that modulates a microbiome biochemical pathway. In some embodiments, the composition is prepared by a process of determining a first metagenome matrix of a microbiome of a subject lacking a target phenotype, determining a second metagenome of a microbiome of a subject possessing the target phenotype, comparing the first and second metagenome matrices, determining a compound that modulates the microbiome biochemical pathway by associating differences between the first and second metagenome matrices to at least one biochemical pathway of the subject wherein the at least one biochemical pathway is associated with the target phenotype, and preparing a composition comprising the compound that modulates the microbiome biochemical pathway.

### EXAMPLES

Some aspects of the embodiments discussed above are disclosed in further detail in the following examples, which are not in any way intended to limit the scope of the present disclosure. Those in the art will appreciate that many other embodiments also fall within the scope of the invention, as it is described herein above and in the claims.

### Example 1

### Methods of Analyzing Skin Microbiota and Metabolic Prediction

The following example demonstrates a method for analyzing skin microbiota and determining metabolome compound community.

Fastq sequencing files, along with the related metadata were collected after consent to participate. Individuals were phenotyped using dual-chamber olfactometer assays to the An. gambiae mosquito (Verhulst et al. 2013; Verhulst et al. 2011; Verhulst et al. 2013). Sequencing was accomplished by 454 pyrosequencing of the V2 region. Closed-reference operational taxonomic unit (OTU) picking was then demultiplexed, trimmed, quality filtered, and completed (with 97% identity to Greengenes) using QIIME v1.9 (Caporaso et al. 2010), yielding an average length of 248 bp (3.23 SD) and 3472.6 sequences per sample (1262.7 SD). PICRUSt (Langille et al. 2013) was then used to predict metagenomes. PICRUSt requires samples to have closed reference picking (annotating 16S rRNA regions to a database of known bacteria) and normalizes copy numbers prior to predicting metagenomes. Next, compounds being consumed or accumulated were determined and termed MetCon scores.

MetCon scores were calculated by creating the metabolome compound community (M) to represent the functional or putatively functional gene pathways. M is a conditional probability matrix of enzymes given the compounds and includes enzymes actively transforming and catalyzing processes and their reactants and metabolites. Metagenomes were predicted from the PICRUSt metagenome prediction rather than direct genetic sequencing. PICRUSt provides a list of KEGG orthology (KO) numbers which were first translated into Enzyme Commission (EC) numbers to be used in MetCon using translation tables available at KEGG. Next, a vector of counts from the translated predicted EC numbers (G) was created. G is quantile normalized and then log₂ transformed. MetCon score were obtained by multiplying matrices M by G to obtain a weighted score of turnover per metabolite. Scores that were negative represent the consumption (input) of a particular metabolite, and positive scores represent the accumulation (output) of a particular metabolite. The consumption of metabolites (input compounds) produce the output metabolites or compounds of interest.

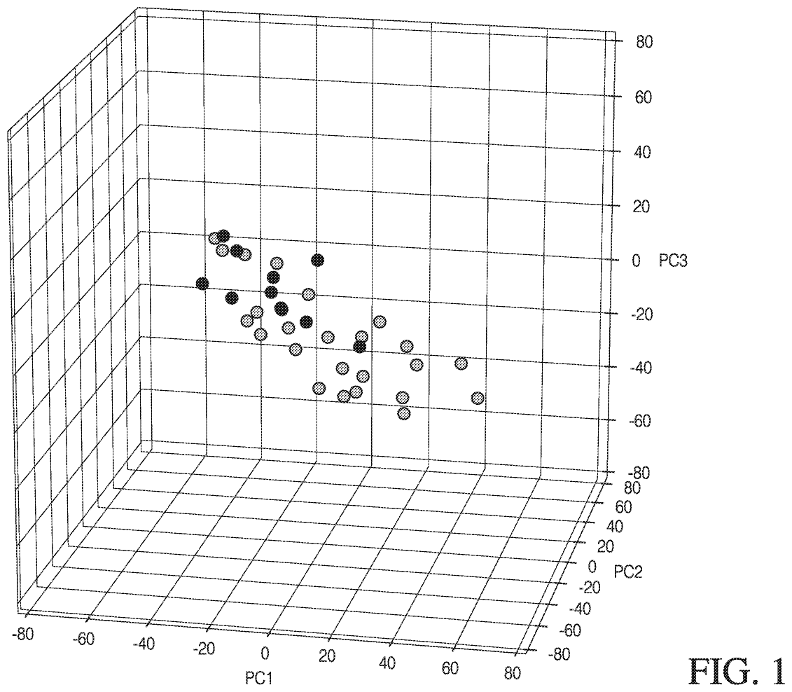

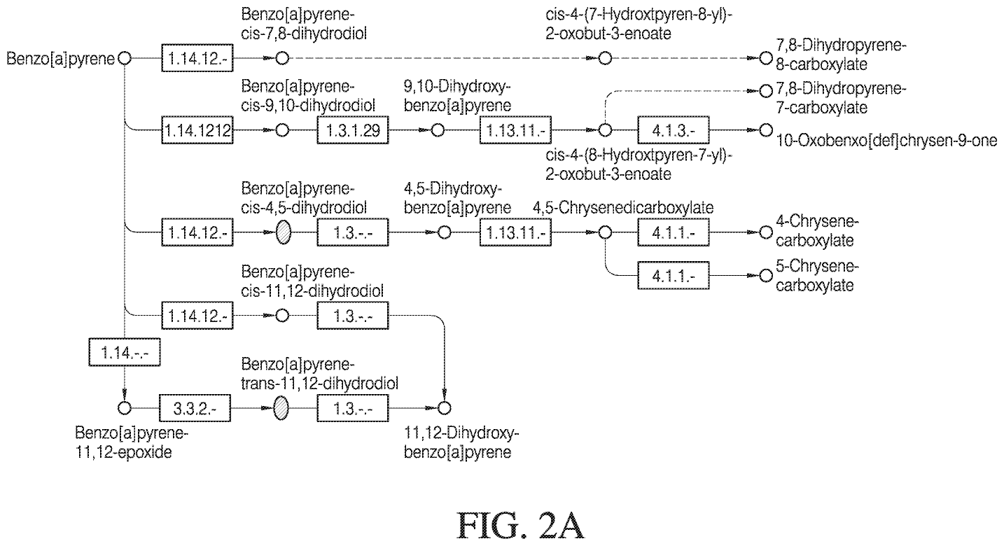

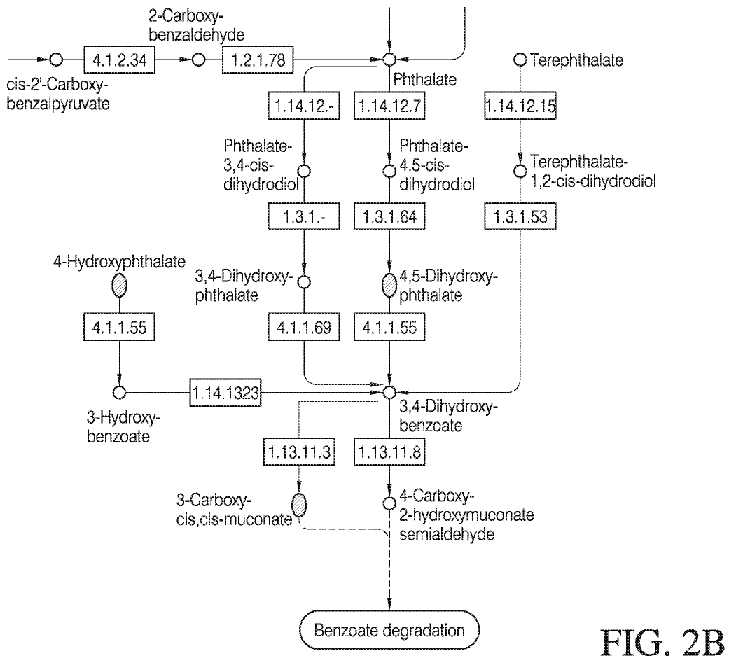

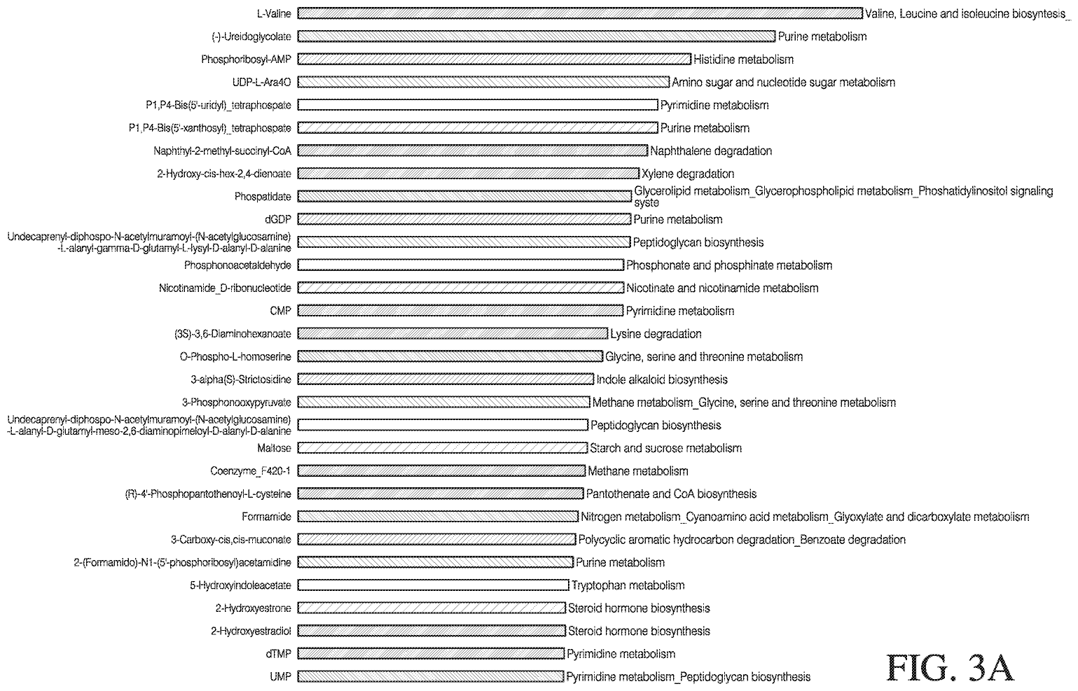

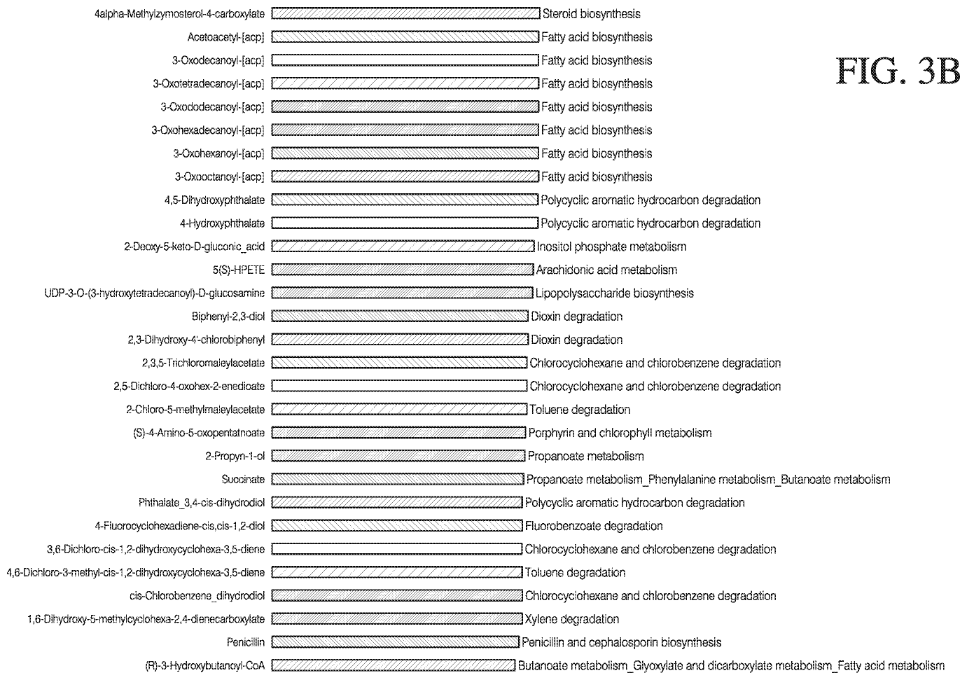

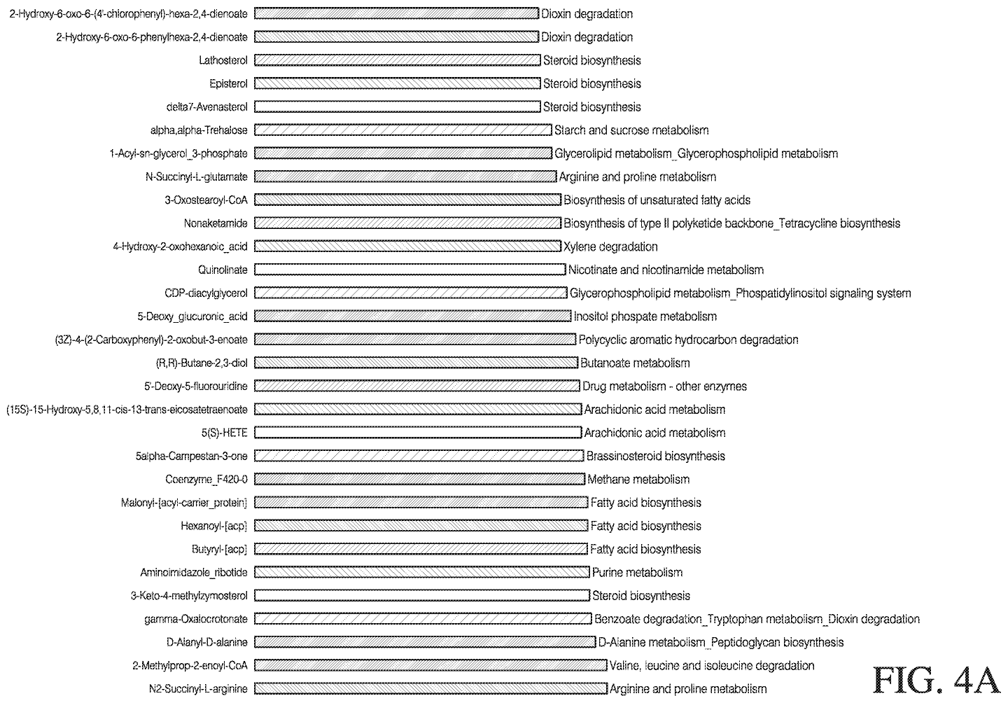

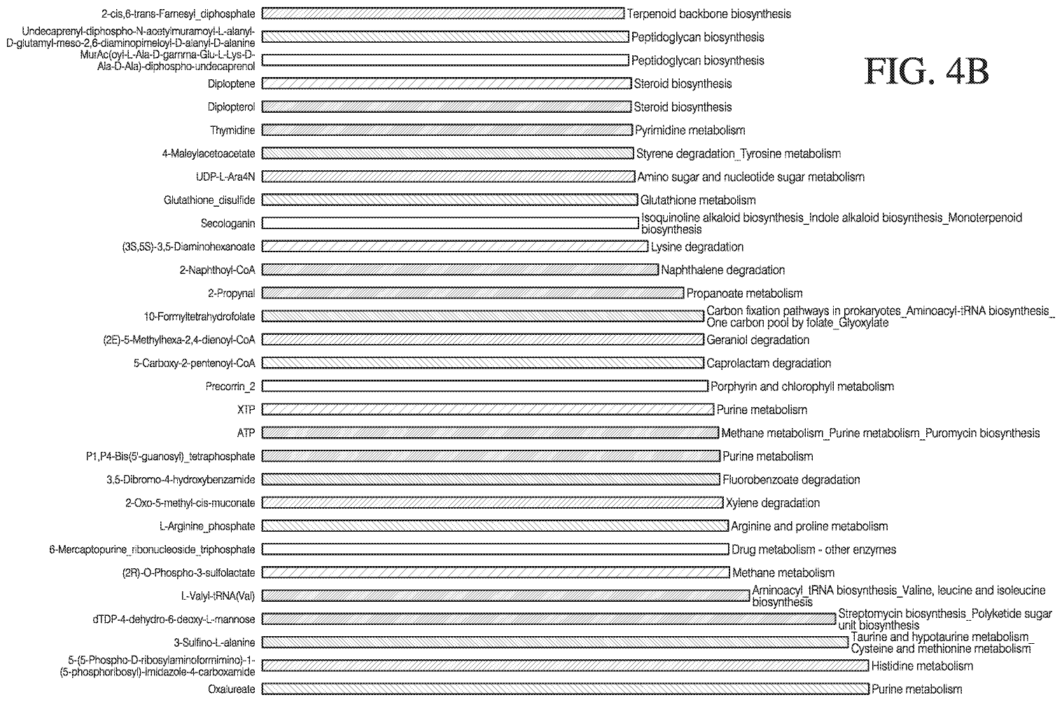

MetCon scores were averaged within the repellent and attractant phenotypes and the difference between the score from the repellent and attractant scores was obtained. Metabolites were annotated to their respective associated pathway using KEGG. To explore gross differences in this metabolome community, scores per sample were composed into a distance matrix, the matrix is centered and decomposed into an eigenmatrix and then the principal coordinates displayed visually (principal coordinates analysis) (FIG. 1). The top 10% of scores were compared to the top 10% scores generated from within organism sample metabolite scores. For comparisons of sample scores, Kruskal-Wallis rank sum tests were used. As shown in FIG. 1, a statistically significant number of the top compound scores had a direct identity to compounds found in the literature. The methods and systems described herein predicted several compounds that were statistically significant, including octanoic acid (p-value=0.01), 1,4-dichlorobenzene (p-value=0.01), benzaldehyde (p-value=0.004), and naphthalene (p-value=0.03) in this data set. In this case metagenomes were predicted and the turnover estimated as per our methods described. FIGS. 3A and 3B depicts the top most accumulated metabolites and their respective pathways by MetCon scores. FIGS. 4A and 4B depicts the top most consumed metabolites and their respective pathways by MetCon scores.

### Example 2

### Scores Calculated Directly from Metagenome Data

The following example demonstrates an exemplary embodiment for determining metagenome scores.

Samples were collected from three body sites (ankle, arm, neck) with three replicates at each site from individuals using sterile cotton swabs. Samples were extracted using QIAamp DNA Microbiome Kit. All samples were de-identified, and random identifiers known as cual-ids, correctable, universally unique identifiers (Chase et al. 2016) were used to identify samples and metadata. Minimum Information about any (x) Sequence checklists (MIxS) standards were complied with on metadata collection (Yilmaz et al. 2011). All basic demographics including age, ancestry, and sex were collected. Participants were assessed for their skin attractiveness to mosquitos by self-report and also quantitative assay. For the quantitative assay, olfactometer bioassays were used to determine skin attractiveness. Standard methods (Qiu et al. 2006; Verhulst et al. 2011; Verhulst et al. 2009) were employed, described here briefly. Skin microbiome samples were collected on glass beads by rubbing 6-10 glass beads on the ankle, arm or leg (as appropriate depending on host organism being tested) and neck (same areas that microbiome swab samples were obtained). The beads were tested for attractiveness to female An. gambiae in a dual-choice olfactometer against a standard ammonia concentration of 136 ppm for six times: two consecutive assays on each of three mornings. Release of test stimuli were alternated between left and right ports to rule out any effects due to port position itself. Using this test “relative attractiveness” was assessed by the number of mosquitoes caught in the trapping device with the individual's microbiome sample tested divided by the total number of mosquitoes trapped.

Library prep and sequencing: Using the extracted DNA from skin samples, libraries were made using the Kapa HyperPlus® kit (Roche), which simultaneously fragments and tags DNA with sequencing adapters for the Illumina HiSeq®-2500 platform. 151 bp paired end sequencing and an insert size of 300 bp were used for sequencing. Prior research shows that the skin microbiome's genome size has a large variance, with an average genome size of 5.5 kb. Thus, the number of samples per lane and estimate sequence coverage that allows for >16kx coverage per genomes of approximately 5.5 kb per sample. Approximately four lanes with about 25 samples per lane were used, including three sample replicates, and three library prep replicates (from a single sample) per lane to assess quality control and technical variation. Replicate samples were sequenced separately and in different lanes.

Shotgun metagenomics allow for the sequencing of all organisms in a sample. This allows for both the prediction of function and composition within samples, and to directly examine the compounds that are different between communities existing on the skin in individuals with a repellency to mosquitos and those that are highly attractive to mosquitos. In samples that have been depleted for host DNA, approximately 10M reads per sample were performed in a sequencing lane. This requires both assembly and direct database annotation. Sequences were preprocessed by removing cloning vector sequences, quality trimming removing low-quality bases, and screening to remove verifiable sequence contaminants (Kunin et al. 2008). Assembling these data without the vector trimming step can produce chimeric contigs where the common vector sequence assembles unrelated sequences.

For draft genome assembly, metaSPAdes (Nurk et al., 2017) was used, which employs efficient assembly graph processing that utilizes rare variants and includes error-correcting and is based on SPAdes (Bankevich et al., 2012). For each scaffold, properties such as the GC content, coverage, genetic code, and profile of phylogenetic affiliation was assessed based on the best hit for each gene in Uniref90 (Suzek et al., 2015). On the basis of analyses of these data, as well as emergent self-organizing map (ESOM)-based analyses of tetranucleotide frequencies and time series relative abundance, draft genomes were generated that include scaffolds from multiple samples (Dick et al., 2009; Sharon et al., 2013). Scaffolds for the same genome found in different samples were aligned to yield longer fragments, leveraging the observation that fragmentation of assemblies is dependent on the context (community composition). Bowtie was used for read mapping (Langmead et al., 2009). Paired-read information was used to extend and join contigs and to fill gaps by the assembler (Sharon et al., 2013).

Although assembly is a useful method for sample composition, it limits the ability to examine low abundance microbes that would be suppressed. Because the goal is to understand the components necessarily driving the community shifts, genes for function were also directly annotated. To do this alignment to reference genomes was performed using shotgun community profiling, MetaPhlAn (Segata et al., 2012) and Centrifuge (Kim et al., 2016) for read-mapping, and additional functional abundance annotations from HUMAnN2 (Abubucker et al., 2012). Enzyme commission (EC) abundances were gathered from the functional abundances, quantile normalized, and then log₂ transformed before analysis. Minimum Information about any (x) Sequence checklists (MIxS) that were established to store metadata for samples (Yilmaz et al., 2011). For HLA typing, HLA*PRG (Dilthey et al., 2016) was used to infer allele type from the metagenome data because host DNA was part of the sequencing results.

The metabolome compound community (M) represents the functional or putatively functional gene pathways. M is the conditional probability matrix of enzymes given the compounds and includes enzymes actively transforming, catalyzing processes and their reactants and metabolites. M was created from KEGG (Kanehisa and Goto 2000). G is the matrix or vector containing the counts of genes related to enzymes or catalyzing processes in the metabolome compound community. G was calculated using the EC sample abundances from the annotation matrix, as described above, and multiplied by G to get a weighted score of turnover per metabolite. Consumption of metabolites was determined to produce the community of interest. Scores were then averaged within the repellent and attractant phenotypes and then the difference between the score from the repellent and attractant scores was calculated. Negative scores represented the consumption of a metabolite, and positive scores represented the accumulation or production of a metabolite. These values can switch depending on whether sample gene values are subtracted prior to multiplication with M, or due to other analysis techniques. Metabolites were also annotated to their respective associated map pathway.

Within a sample organism, scores were calculated similarly as above, but with several differences. G was multiplied by the matrix of per sample, per organism fractional probabilities of abundance, prior to multiplication by M. This resulted in a per sample per organism compound scores. Annotated gene abundances per sample per organism were found using the assembled sequence. The top 10% of scores were compared to the top 10% scores generated from within organism sample metabolite scores. For comparisons of sample scores, Kruskal-Wallis rank sum tests were used, where applicable.

To create models for the repellent and attractive community, the compounds and organisms creating those compounds, the organisms were used to create an interaction network, and this network was represented as a set of explicit relationships inferred from the predicted compound data. The interaction network was a generation of a Bayesian inference network of microorganism assemblages as a directed acyclical graph (DAG), in which the parent nodes were changes in environmental parameters over time and space; the daughter nodes were changes in the relative abundance of the community. The environmental parameters were the compounds and their mass estimated from mass spectrometry methods. Directed edges between nodes indicated correlations. Such networks can be generated with standard software that implements Bayesian network inference (such as the BayesPy Python package). For representing the network, the value of the nodes were expressed as a function of the value of its parent nodes. This was addressed by standard tools of unknown response surface learning, such as artificial neural network (ANN) tools, a form of artificial intelligence methods. These generated ANNs represent microbial community structure in terms of mathematical equations that best explain the data, and were used to predict the relative abundance of taxa in time or space as functions of environmental conditions. These ANNs capture potentially causal relationships between the changing abundances of different taxa, although relationships between taxa could arise through taxon proxies for changes in environmental parameters.

### Example 3

### Analysis of Compounds from Skin Emanations

The following example demonstrates an exemplary embodiment for measuring compounds from skin emanations using gas chromatography mass spectrometry (GCMS).

Subjects were asked to rub the sole of their left foot against glass beads for 10 minutes. The beads were then be distributed to GCMS autosampler vials equipped with a pierceable rubber gasket and placed at −20° C. until analysis to prevent desorption of adsorbed volatiles. Glass beads have the advantage as a collection device that adsorbed skin emanations can be removed for GCMS analysis through thermal desorption (Bernier et al., 1999). GCMS was only tested on a subset of samples from individuals; only those individuals described as having ‘highly attractive’ and ‘poorly attractive’ cohorts based on the number of mosquitoes that make oriented movements toward or away from the port containing glass beads bearing human skin emanations will have their skin volatiles tested.

Emanations were analyzed using thermal desorption followed by GCMS. The system included a thermal desorption autosampler, an electrically-cooled trap for focusing, and a flow controller for thermal desorption injection into a Trace GC Ultra (Thermo Scientific, USA) coupled to a quadruple mass detector (DSQ, Thermo Scientific, USA). The cartridges were dry-purged for one minute with helium (5.0 grade) at 30° C. to remove residual moisture and oxygen. Cartridges were desorbed at 150° C. for 10 min and the volatiles focused on an electronically cooled sorbent trap (general purpose hydrophobic, Markes, UK) at −10° C. The transfer line between the GC and MS was maintained at 275° C. The column effluent was ionized by electron impact at 70 eV and mass-spectra were recorded in positive mode from 35-300 m/z with a scan speed of five scans/s and an ion source temperature of 250° C.

Volatile profiles were screened against a reference library of compounds that were identified in previous skin emanation studies of mosquito attraction (Verhulst et al., 2009; Smallegange et al., 2009; Logan et al., 2008), and by using the publically available NIST GCMS reference library. Compounds were identified by comparing their mass spectra and retention times with those of authentic reference compounds. Relative quantification of the compounds were based on characteristic mass ions for each compound using the software package Xcalibur (Thermo Scientific) with peak deconvolution by AMDIS. The obtained spectra were compared to the NIST-library. Calculated and reported retention indices and injection of authentic synthetic reference compounds provided additional information for identification. Overlaying the observed GCMS compounds with a pathway enrichment analysis (from annotated-assembled data) allow categorization of biochemical pathways most associated with a mosquito-repellent microbiome and those most associated with mosquito attraction.

The GCMS results indicated agreement between the GCMS molecules observed in the highly attractive and poorly attractive cohorts with the in silico output predictions (associated with predicted candidate input compounds) described in Example 2. Thus, the methods of Example 2 are useful for predicting the compounds output by microbial metabolisms on the skin, and by mathematical relationship between the input and output predicted compounds. The GCMS results also resulted in common chemical mediators of An. gambiae attraction. These attractants have received more focus than endogenous microbiome repellent capacity, including: lactic acid, aliphatic carboxylic acids (propanoic acid, butanoic acid, 3-methylbutanoic acid, pentanoic acid, heptanoic acid, octanoic acid, and tetradecanoic acid) and 3-methyl-1-butanol (Bernier et al., 1999; Braks et al., 2001; Busula et al., 2017; Daisy et al., 2002; G Logan et al., 2008). The microbial biochemical pathways involved in the generation of these chemical entities were more highly represented in the output metabolites from metagenomic sequencing data outlined in Example 2.

### Example 4

### Analysis of Insect Repellency in Animals

The following example demonstrates an exemplary embodiment for measuring insect repellency in animals.

In addition to humans, differential attraction to insects has long been noted in animals. Five times more ticks were found on cocker spaniels than on beagles. To collect skin and hair samples, a clean piece of flannel was rubbed on the dogs for 15 min and these were tested for arrestment and attractiveness of ticks. Three choices were offered: cocker extract vs. control; beagle extract vs. control, and cocker extract vs. beagle extract. When allowed to choose between substances rubbed from dogs and a control, more ticks were arrested by extracts from the cockers than from beagles. In the arrestment tests with only a choice between substances from dogs of each breed, more ticks were arrested by cocker substances. To test for attraction, capsules containing adsorbent were used and the tests were carried out in a Y-olfactometer. Fifteen males and 15 females were tested, for each treatment. R. sanguineus can use substances from the dogs to differentiate susceptible English Cocker Spaniels from resistant Beagles. (Louly et al., 2010). In comparing beagles to other non-resistant dogs benzaldehyde, 2-hexanone, undecane, decane and nonane are compounds produced via the microbiome, as well as even-numbered saturated and monounsaturated aldehydes and octanal. Control of ticks such as R. sanguineus (s.l.) is still predominantly achieved by using acaricides that act as broad-spectrum neurotoxins. Overuse of these agents has led to emergence of acaricide resistance and presence of strains of R. sanguineus (s.l.) that are resistant to commercially available acaricides has been recorded in various parts of the world.

Like dogs, certain cattle breeds have been found to be more attractive or repellent to insects. The tick Rhipicephalus microplus specializes on cattle and other large bovids. However, success of its blood-feeding depends on the breed of the bovine host. In an indicine and a taurine breed of cattle that present contrasting phenotypes of infestation, Nelore and Holstein, respectively, resistant and susceptible to tick infestations as ascertained by the number of ticks and the reproductive success of female ticks completing their life-cycles on these two types of host. Taurine breeds suffer debilitating infestations with hundreds of feeding parasites, whereas indicine breeds typically exhibit few engorging females that lay smaller batches of eggs than females fed on susceptible hosts. These contrasting tick burdens are highly heritable (Wambura et al., 1998) and offer a useful model to study the mechanisms that result in resistance to blood-feeding ectoparasites.

At the same time, different levels of host immunity may affect the composition of tick saliva, contributing to these outcomes. In order to understand the different host defense mechanisms that control hematophagous ectoparasites and result in different tick loads, the following measures are analyzed.

1. Tick bites induce changes in gene expression profiles in the skin of their hosts that highlight the proteins and defense pathways that participate in skin reactions to ticks.

2. Relative to skin from animals of a tick-susceptible breed of cattle, skin from animals of a tick-resistant breed provide baseline and reactive expression profiles of genes that indicated the proteins and defense pathways involved in repelling or expelling ticks more efficiently from the host's skin.

3. Differences in the local reaction to bites in resistant and susceptible hosts will affect expression of genes encoding secreted salivary proteins of the tick that mediate parasitism.

The produced compound 6-methyl-5-hepten-2-one was determined to be repellent. Genes including ALDH1A1 (AL1A1), SULT1A1, DNAJC12, SCARA5, Bt.23579 LOC785756, Bt.19274 (C1QTNF7), AKR1C2, Bt.21056 (DERL1), SAA3, CERS4, AKR1C3 (Bt.63212), SCG2, NR4A2, TOB1, HMGB1, IFI6, CSTD, Pseudogene (HNRNPK), HNRNPK, IL-3, EIF2AK2 (PKR), SRRM2, NFAT5, Bt.25055 (IPMK), CHD4, Bt.95322 (MARCKS), AEBP2, EGFR, MGC155143, JUN, ISG15, PNRC2, FZD10, PDPK1, ARPC3, IDH1, Pseudogene IDH1 were each differentially expressed in the host.

### Example 5

### Analysis of Oral Health Microbiome in Humans

The following example demonstrates an exemplary embodiment for measuring periodontal disease and oral health from analyzing the oral microbiome of humans.

Caries samples included teeth with dentinal caries lesions that resulted in pulp exposure and were diagnosed with symptomatic irreversible pulpitis on the basis of clinical and radiographic findings, according to the reports of the American Association of Endodontists Consensus Conference on diagnostic terminology. Pulp vitality was confirmed by thermal sensibility tests. Radiographic analysis showed extensive caries lesions, mature root apexes, and normal width of the apical periodontal ligament space. Teeth presenting with necrotic pulps or treated root canals, as well as teeth with no evidence of pulp exposure following caries removal, were excluded from the study. Individuals exhibited no evidence of marginal periodontitis and reported no significant systemic condition. The caries sample material was placed in cryotubes containing Tris-EDTA buffer (10 mM Tris-HCl, 1 mM EDTA, pH 7.8) and immediately frozen at −20° C. DNA was extracted from caries dentin samples by using the QIAamp DNA Mini Kit (Qiagen, Valencia, Calif., USA). For healthy plaque samples, subjects were included if they were as follows: in good general health, ≥35 years, ≥15 teeth and had the absence of active caries lesions, orthodontic appliances, previous periodontal treatment, antibiotic use or continual use of mouthwashes containing antimicrobials in the previous six months. Samples were collected individually with Gracey curettes and placed in a microtube with 150 μL TE buffer. Afterwards, the MasterPure™ Complete DNA and RNA Purification Kit protocol was followed (MC85200, Epicenter, USA). DNA concentration was measured individually with a Qubit™ 2.0 fluorometer (Thermo-Fisher-Scientific Inc., USA).

The 16S rRNA gene V4 variable region primers 515/806 with barcode on the forward primer were used in a 30-cycle polymerase chain reaction (PCR) assay using the HotStarTaq Plus Master Mix Kit (Qiagen) under the following conditions: 94° C. for 3 minutes, followed by 28 cycles of 94° C. for 30 seconds, 53° C. for 40 seconds and 72° C. for 1 minute, and then a final elongation step at 72° C. for 5 minutes. For healthy samples, they were amplified via a two-step PCR approach. Samples were pooled together in equal proportions based on their molecular weight and DNA concentrations. Pooled samples were purified using Ampure XP beads. Pooled and purified PCR product was used to prepare DNA library by following Illumina TruSeq DNA library preparation protocol. Paired-end sequencing was performed on Illumina MiSeq device (Illumina Inc., San Diego, Calif., USA) following the manufacturer's guidelines.

Caries active sites have included metabolites isooctanol (Rt 26.52 min), 2-ethyl-1-hexanol acetate (Rt 28.74), 3-methyl-1-heptanol or 6-methyl-1-heptanol (Rt 29.49 min), 2-propenoic acid octyl ester (Rt 32.38 min), 4,8-dimethylnonanol by GCMS analysis. In general alcohol esters were increased in active caries groups.

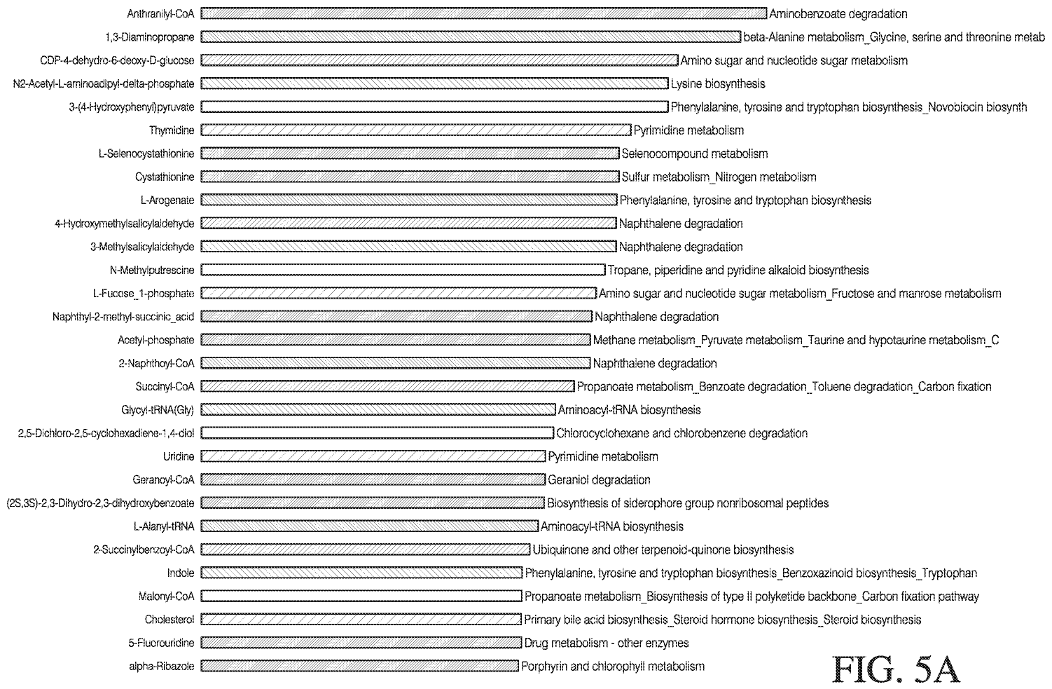

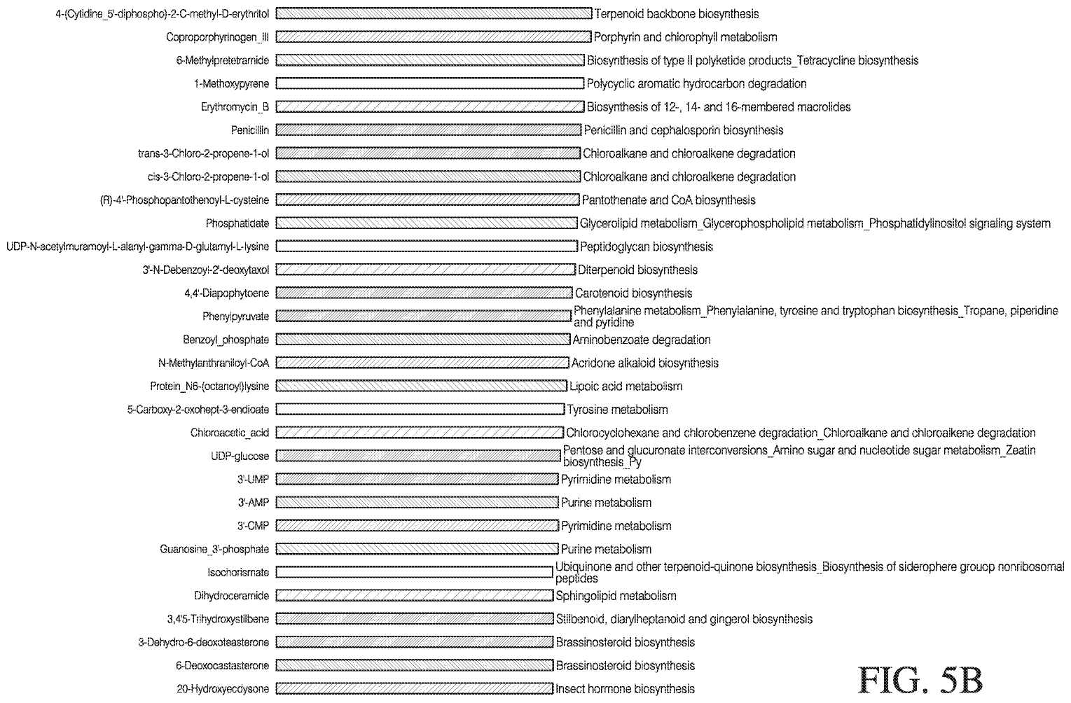

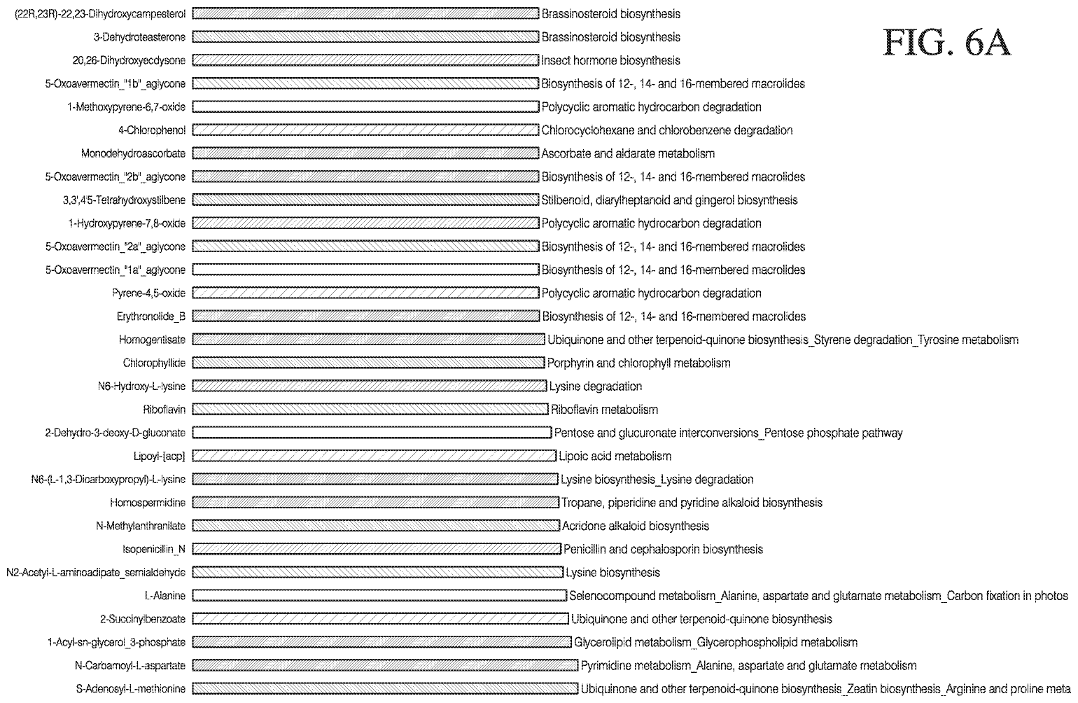

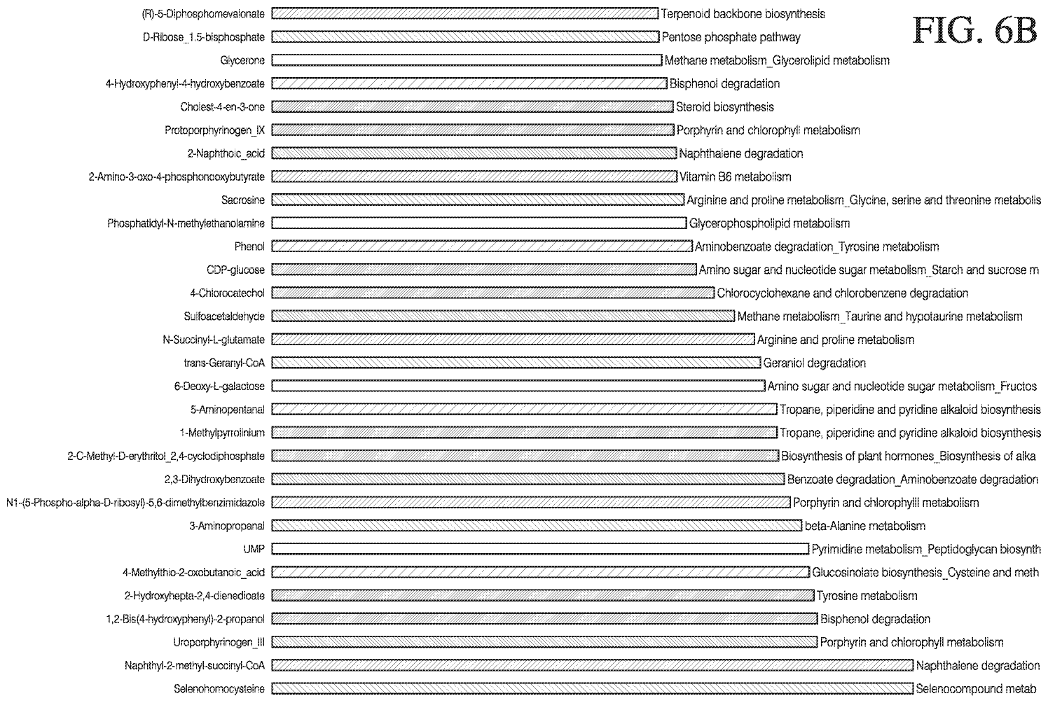

Caries and normal healthy plaque data from both studies was downloaded from EMBL as fastq files, then demultiplexed, trimmed, quality filtered and completed closed-reference OTU picking (with 97% identity to Greengenes) using QIIME v1.9 (Caporaso et al., 2010), yielding an average length of 276 bp (25 SD) and 263923.5 sequences per sample (453984.9 SD). Next, PICRUSt (Langille et al., 2013) was used to predict metagenomes. PICRUSt requires samples to have closed reference picking (annotating 16S rRNA regions to a database of known bacteria) and normalizes copy numbers prior to predicting metagenomes, (otherwise non-OTU (amplicon sequence variant (ASV) methods) and QIIME 2.0 would have been used. Next, the methods described herein were used to determine the compounds being accumulated (output; FIGS. 5A and 5B) or consumed (input; FIGS. 6A and 6B).

### Example 6

### Atopic Dermatitis Microbiome

The following example demonstrates an exemplary embodiment for measuring the microbiome in subject with atopic dermatitis as compared to healthy individuals.

Two swab samples were collected from the forearm of each patient with AD, one from lesional skin and one from adjacent normal-appearing non-lesional skin. All samples were collected from a 5 cm×5 cm area. AD was diagnosed with standard criteria based on the American Academy of Dermatology and the NIH/NIAID Atopic Dermatitis Research Network. Disease severity was assessed by using the Rajka-Langeland scoring system, which rates extent, course, and itch intensity separately. Healthy subjects were defined as having no personal or family history of atopic diseases and no personal history of chronic skin or systemic diseases. V1-V3 of the 16S rRNA genes were amplified from purified genomic DNA using primers 27F and 534R. PCR amplification was performed according to the protocol developed by the Human Microbiome Project. 16S rRNA amplicon libraries were purified, quantified using qPCR and pooled for sequencing on Illumina MiSeq platform (Illumina, Inc., San Diego, Calif.).

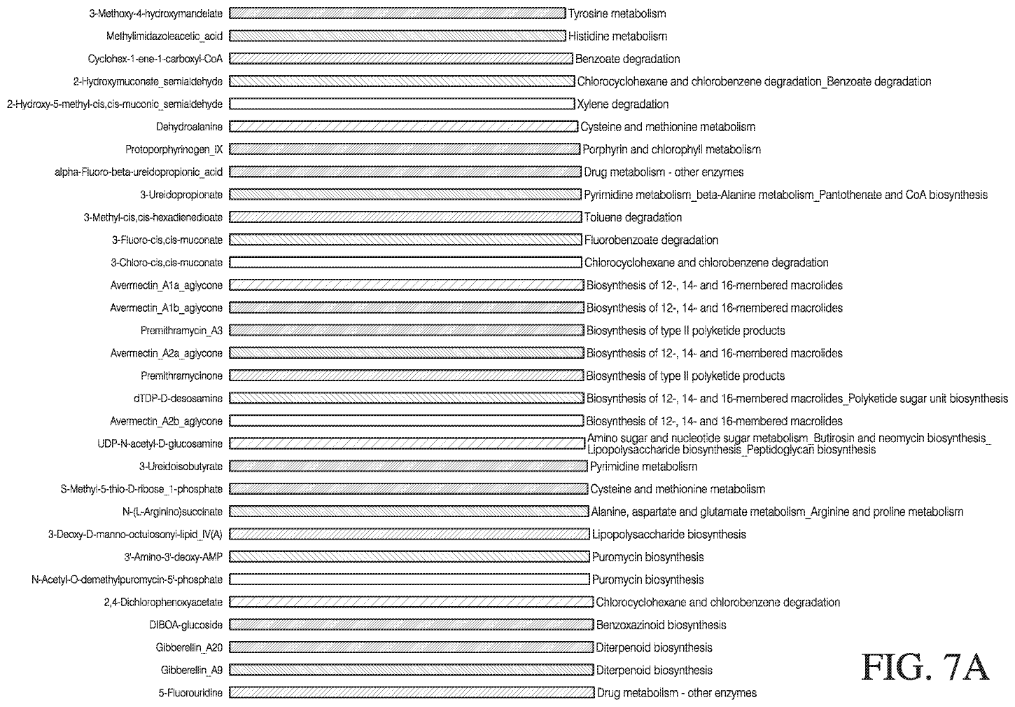

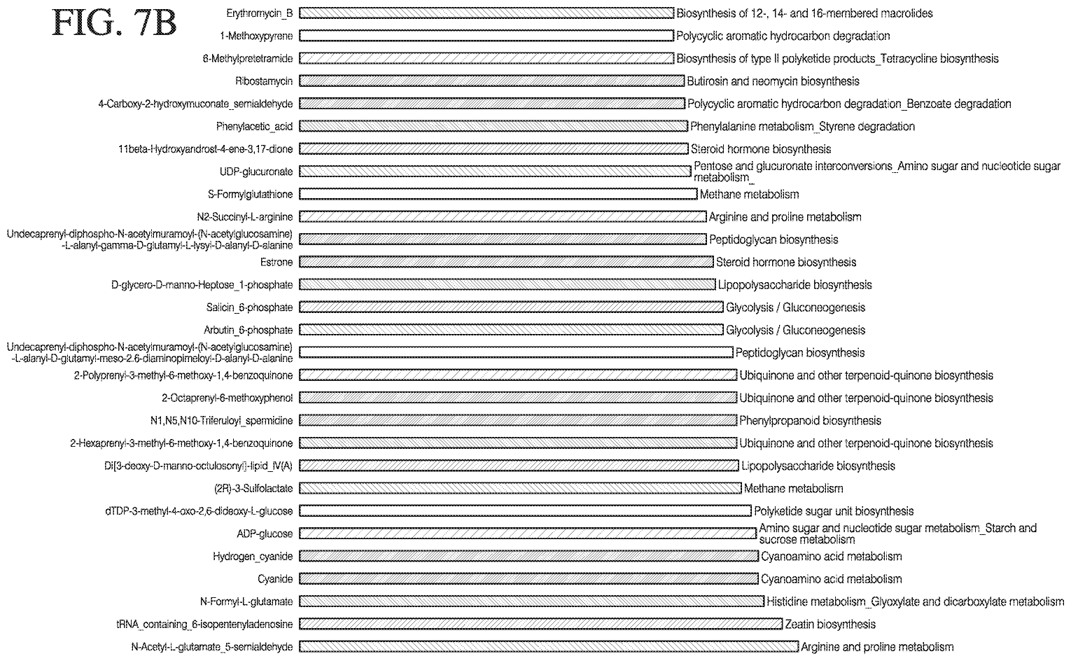

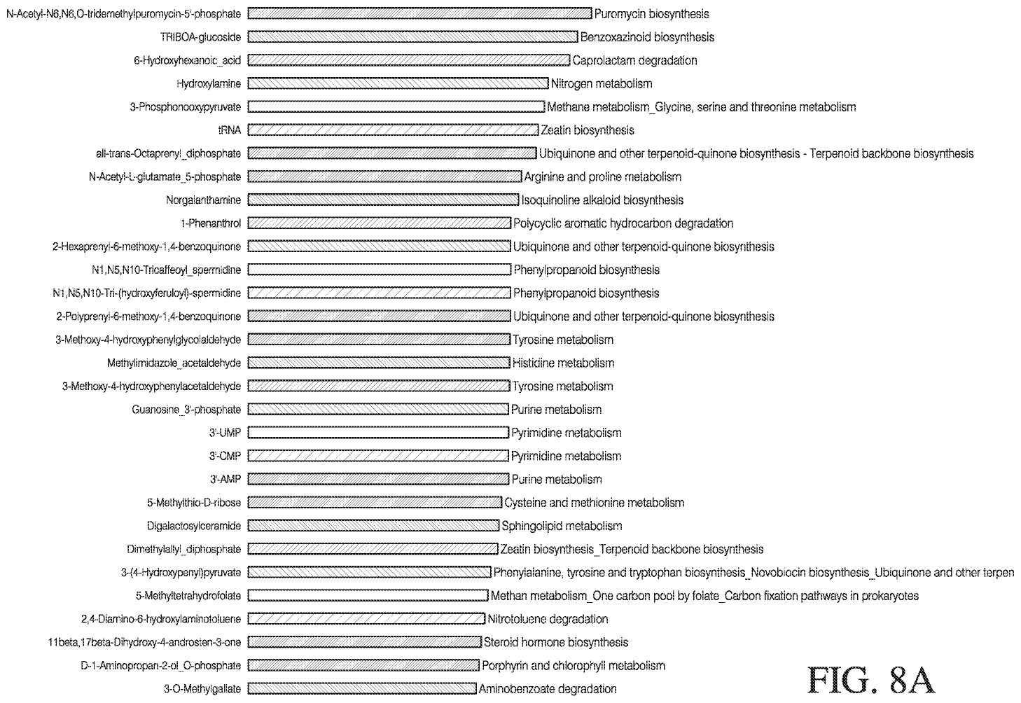

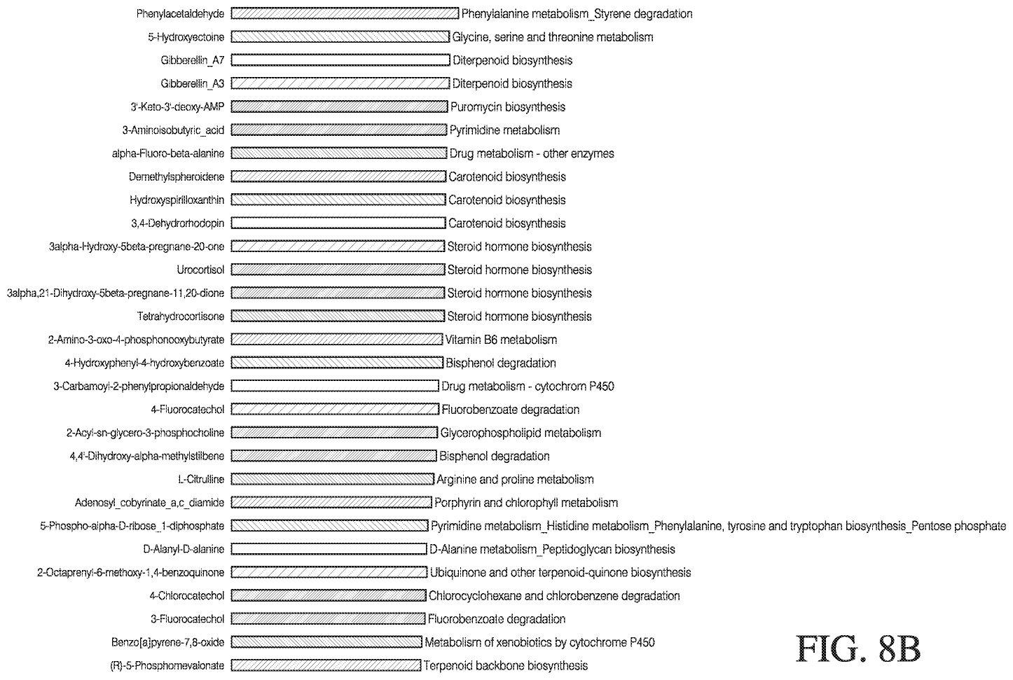

The data from atopic and non-atopic lesions was downloaded from EMBL as fastq files, then demultiplexed, trimmed, quality filtered and completed closed-reference OTU picking (with 97% identity to Greengenes) using QIIME v1.9 (Caporaso et al., 2010). Next, we used PICRUSt (Langille et al., 2013) to predict metagenomes. Next, the methods described herein were used to determine the compounds that were consumed (FIGS. 7A and 7B) or accumulated (FIGS. 8A and 8B).

### Exemplary Methods and Compositions

Any of the features of the following sixty six exemplary methods and composition is applicable to all aspects and embodiments identified herein, including other of the sixty six exemplary methods and compositions. Moreover, any of the features of the following sixty six exemplary methods and compositions is independently combinable, partly or wholly with other aspects and embodiments described herein in any way, e.g., one, two, or three or more embodiments may be combinable in whole or in part, including in connection with any of the sixty six exemplary methods and compositions. Further, any of the features of the following sixty six exemplary methods and compositions may be made optional, including to other of the sixty six exemplary methods and compositions. Any aspect or embodiment of a method can be performed by a composition of another aspect or embodiment, and any aspect or embodiment of a composition can be configured to perform a method of another aspect or embodiment, including in connection with the sixty six exemplary methods and compositions.

Exemplary Method No. 1: A method for identifying one or more biochemical pathways associated with a phenotype of a dermal sample comprising: determining a first metagenome matrix of a dermal sample of at least one subject lacking a phenotype; determining a second metagenome of a dermal sample of at least one subject possessing a phenotype; comparing the first and second metagenome matrices; and associating differences between the first and second metagenome matrices to at least one biochemical pathway of the subject wherein the at least one biochemical pathway is associated with the phenotype.

Exemplary Method No. 2: The method of Exemplary Method No. 1, wherein the phenotype comprises insect repellence.

Exemplary Method No. 3: The method of Exemplary Method No. 1, wherein the phenotype comprises insect attraction.

Exemplary Method No. 4: The method of any one of Exemplary Method Nos. 1 to 3, wherein the phenotype comprises a dermal metabolite.

Exemplary Method No. 5: The method of any one of Exemplary Method Nos. 1 to 4, wherein the dermal sample is selected from the group consisting of: skin, hair, and fur.

Exemplary Method No. 6: The method of any one of Exemplary Method Nos. 1 to 5, further comprising identifying at least one agent that is a compound of the at least one biochemical pathway.

Exemplary Method No. 7: The method of any one of Exemplary Method Nos. 1 to 6, wherein the at least one biochemical pathway is selected from the group consisting of: Xylene degradation; Glucosinolate biosynthesis; Aminoacyl-tRNA biosynthesis; Penicillin and cephalosporin biosynthesis; Valine degradation; Leucine degradation; Isoleucine degradation; Tryptophan metabolism; Steroid biosynthesis; Starch and sucrose metabolism; Pyrimidine metabolism; Purine metabolism; Propanoate metabolism; Butanoate metabolism; Citrate cycle; Tyrosine metabolism; Phenylalanine metabolism; caprolactam degradation or metabolism; Toluene degradation; Glyoxylate metabolism; Dicarboxylate metabolism; Alanine metabolism; Aspartate metabolism; Glutamate metabolism; Propanoate metabolism; Porphyrin metabolism; Chlorophyll metabolism; Polycyclic aromatic hydrocarbon degradation; Benzoate degradation; Phosphonate metabolism; Phosphinate metabolism; Peptidoglycan biosynthesis; Penicillin and cephalosporin biosynthesis; Pantothenate and CoA biosynthesis; Nitrogen metabolism; Cyanoamino acid metabolism; Nicotinate and nicotinamide metabolism; Naphthalene degradation; Methane metabolism; Glycine metabolism; serine metabolism; threonine metabolism; Lysine degradation; Lipopolysaccharide biosynthesis; Inositol phosphate metabolism; Indole alkaloid biosynthesis; Histidine metabolism; Glycerolipid metabolism; Glycerophospholipid metabolism; Phosphatidylinositol signaling system; Fluorobenzoate degradation; Fatty acid biosynthesis; Dioxin degradation; Chlorocyclohexane and chlorobenzene degradation; Butanoate metabolism; Fatty acid metabolism; Arachidonic acid metabolism; Amino sugar metabolism; nucleotide sugar metabolism; vitamin B6 metabolism; geraniol degradation; cintronella degradation; limonene degradation; and pinene degradation.

Exemplary Method No. 8: The method of any one of Exemplary Method Nos. 1 to 7, wherein the determining of the first or second metagenome matrix is obtained from one or a plurality of subjects.

Exemplary Method No. 9: The method of any one of Exemplary Method Nos. 1 to 8, wherein the metagenome matrix comprises a metatranscriptome.

Exemplary Method No. 10: The method of any one of Exemplary Method Nos. 1 to 9, further comprising identifying at least one agent that modulates the gene expression of at least one component for the at least one biochemical pathway.

Exemplary Method No. 11: A method for identifying one or more biochemical pathways associated with a phenotype of a dermal sample comprising: determining a first metabolome compound matrix of a dermal sample of at least one subject lacking a phenotype; determining a second metabolome compound matrix of a dermal sample of at least one subject possessing a phenotype; comparing the first and second metabolome compound matrices; and associating differences between the first and second metabolome compound matrices to at least one biochemical pathway; wherein the at least one biochemical pathway is associated with the phenotype.

Exemplary Method No. 12: The method of Exemplary Method No. 11, wherein the phenotype comprises insect repellence.

Exemplary Method No. 13: The method of Exemplary Method No. 11, wherein the phenotype comprises insect attraction.

Exemplary Method No. 14: The method of any one of Exemplary Method Nos. 11 to 13, wherein the phenotype comprises a dermal metabolite.

Exemplary Method No. 15: The method of any one of Exemplary Method Nos. 11 to 14, wherein the dermal sample is selected from the group consisting of skin, hair, and fur.

Exemplary Method No. 16: The method of any one of Exemplary Method Nos. 11 to 15, further comprising identifying at least one agent that is a compound of the at least one biochemical pathway.

Exemplary Method No. 17: The method of any one of Exemplary Method Nos. 11 to 16, wherein the at least one biochemical pathway is selected from the group consisting of: Xylene degradation; Glucosinolate biosynthesis; Aminoacyl-tRNA biosynthesis; Penicillin and cephalosporin biosynthesis; Valine degradation; Leucine degradation; Isoleucine degradation; Tryptophan metabolism; Steroid biosynthesis; Starch and sucrose metabolism; Pyrimidine metabolism; Purine metabolism; Propanoate metabolism; Butanoate metabolism; Citrate cycle; Tyrosine metabolism; Phenylalanine metabolism; caprolactam degradation or metabolism; Toluene degradation; Glyoxylate metabolism; Dicarboxylate metabolism; Alanine metabolism; Aspartate metabolism; Glutamate metabolism; Propanoate metabolism; Porphyrin metabolism; Chlorophyll metabolism; Polycyclic aromatic hydrocarbon degradation; Benzoate degradation; Phosphonate metabolism; Phosphinate metabolism; Peptidoglycan biosynthesis; Penicillin and cephalosporin biosynthesis; Pantothenate and CoA biosynthesis; Nitrogen metabolism; Cyanoamino acid metabolism; Nicotinate and nicotinamide metabolism; Naphthalene degradation; Methane metabolism; Glycine metabolism; serine metabolism; threonine metabolism; Lysine degradation; Lipopolysaccharide biosynthesis; Inositol phosphate metabolism; Indole alkaloid biosynthesis; Histidine metabolism; Glycerolipid metabolism; Glycerophospholipid metabolism; Phosphatidylinositol signaling system; Fluorobenzoate degradation; Fatty acid biosynthesis; Dioxin degradation; Chlorocyclohexane and chlorobenzene degradation; Butanoate metabolism; Fatty acid metabolism; Arachidonic acid metabolism; Amino sugar metabolism; nucleotide sugar metabolism; vitamin B6 metabolism; geraniol degradation; cintronella degradation; limonene degradation; and pinene degradation.

Exemplary Method No. 18: The method of any one of Exemplary Method Nos. 11 to 17, wherein the determining of the first or second metabolome compound matrix comprises subjecting the dermal sample to an analytical method selected from the group consisting of: a genomics method assessment, a transcriptomic or metabolomics assessment, a determination of microbiome composition, nuclear magnetic resonance (NMR) and mass spectrometry (MS), Fourier-transform infrared (FTIR), infrared (IR) thermography, cataluminescence (CTL), laser-induced fluorescence imaging (LIFT), and resonance-enhanced multiphoton ionization (REMPI).

Exemplary Method No. 19: The method of any one of Exemplary Method Nos. 11 to 17, wherein the determining of the first or second metabolome compound matrix is obtained from one or a plurality of subjects.

Exemplary Method No. 20: A method for identifying one or more metabolites associated with a phenotype of interest comprising: determining a first metabolite profile of a dermal sample of at least one subject lacking a phenotype; determining a second metabolite profile of a dermal sample of at least one subject possessing a phenotype; comparing the first and second metabolite profiles; and identifying at least one metabolite associated with the phenotype.

Exemplary Method No. 21: The method of Exemplary Method No. 20, wherein the phenotype is insect repellence.

Exemplary Method No. 22: The method of Exemplary Method No. 20, wherein the phenotype is insect attraction.

Exemplary Method No. 23: The method of Exemplary Method No. 20, wherein the dermal sample is selected from the group consisting of: skin, hair, fur.

Exemplary Method No. 24: The method of Exemplary Method No. 20, further comprising associating the differences in the first and second metabolite profiles to at least one biochemical pathway of the subject.

Exemplary Method No. 25: The method of Exemplary Method No. 20, wherein the at least one biochemical pathway is selected from the group consisting of: Xylene degradation; Glucosinolate biosynthesis; Aminoacyl-tRNA biosynthesis; Penicillin and cephalosporin biosynthesis; Valine degradation; Leucine degradation; Isoleucine degradation; Tryptophan metabolism; Steroid biosynthesis; Starch and sucrose metabolism; Pyrimidine metabolism; Purine metabolism; Propanoate metabolism; Butanoate metabolism; Citrate cycle; Tyrosine metabolism; Phenylalanine metabolism; caprolactam degradation or metabolism; Toluene degradation; Glyoxylate metabolism; Dicarboxylate metabolism; Alanine metabolism; Aspartate metabolism; Glutamate metabolism; Propanoate metabolism; Porphyrin metabolism; Chlorophyll metabolism; Polycyclic aromatic hydrocarbon degradation; Benzoate degradation; Phosphonate metabolism; Phosphinate metabolism; Peptidoglycan biosynthesis; Penicillin and cephalosporin biosynthesis; Pantothenate and CoA biosynthesis; Nitrogen metabolism; Cyanoamino acid metabolism; Nicotinate and nicotinamide metabolism; Naphthalene degradation; Methane metabolism; Glycine metabolism; serine metabolism; threonine metabolism; Lysine degradation; Lipopolysaccharide biosynthesis; Inositol phosphate metabolism; Indole alkaloid biosynthesis; Histidine metabolism; Glycerolipid metabolism; Glycerophospholipid metabolism; Phosphatidylinositol signaling system; Fluorobenzoate degradation; Fatty acid biosynthesis; Dioxin degradation; Chlorocyclohexane and chlorobenzene degradation; Butanoate metabolism; Fatty acid metabolism; Arachidonic acid metabolism; Amino sugar metabolism; nucleotide sugar metabolism; vitamin B6 metabolism; geraniol degradation; cintronella degradation; limonene degradation; and pinene degradation.

Exemplary Method No. 26: The method of Exemplary Method No. 20, further comprising identifying at least one agent that is a substrate of the at least one biochemical pathway.

Exemplary Method No. 27: A method of modulating a phenotype of a subject by contacting the subject with a compound identified by the method of Exemplary Method No. 6 or Exemplary Method No. 15.

Exemplary Method No. 28: The method of Exemplary Method No. 27, wherein the subject lacks the phenotype.

Exemplary Method No. 29: The method of Exemplary Method No. 27, wherein the subject possesses the phenotype.

Exemplary Method No. 30: The method of any one of Exemplary Method Nos. 27 to 29, wherein the phenotype is insect repellence.

Exemplary Method No. 31: The method of any one of Exemplary Method Nos. 27 to 29, wherein the phenotype is insect attraction.

Exemplary Method No. 32: A method for identifying one or more biochemical pathways associated with a phenotype of a gut sample comprising: determining a first metabolome compound matrix of a gut sample of at least one subject lacking a phenotype; determining a second metabolome compound matrix of a gut sample of at least one subject possessing a phenotype; comparing the first and second metabolome compound matrices; and associating differences between the first and second metabolome compound matrices to at least one biochemical pathway; wherein the at least one biochemical pathway is associated with the phenotype.

Exemplary Method No. 33: The method of Exemplary Method No. 32, wherein the phenotype comprises insect repellence.

Exemplary Method No. 34: The method of Exemplary Method No. 32, wherein the phenotype comprises insect attraction.

Exemplary Method No. 35: The method of any one of Exemplary Method Nos. 32 to 34, wherein the phenotype comprises a gut metabolite.

Exemplary Method No. 36: The method of any one of Exemplary Method Nos. 32 to 35, wherein the gut sample is selected from the group consisting of: esophagus, stomach, small intestine, large intestine, and a fecal material.

Exemplary Method No. 37: The method of any one of Exemplary Method Nos. 32 to 36, further comprising identifying at least one agent that is a compound of the at least one biochemical pathway.

Exemplary Method No. 38: The method of any one of Exemplary Method Nos. 32 to 37, wherein the at least one biochemical pathway is selected from the group consisting of: Xylene degradation; Glucosinolate biosynthesis; Aminoacyl-tRNA biosynthesis; Penicillin and cephalosporin biosynthesis; Valine degradation; Leucine degradation; Isoleucine degradation; Tryptophan metabolism; Steroid biosynthesis; Starch and sucrose metabolism; Pyrimidine metabolism; Purine metabolism; Propanoate metabolism; Butanoate metabolism; Citrate cycle; Tyrosine metabolism; Phenylalanine metabolism; caprolactam degradation or metabolism; Toluene degradation; Glyoxylate metabolism; Dicarboxylate metabolism; Alanine metabolism; Aspartate metabolism; Glutamate metabolism; Propanoate metabolism; Porphyrin metabolism; Chlorophyll metabolism; Polycyclic aromatic hydrocarbon degradation; Benzoate degradation; Phosphonate metabolism; Phosphinate metabolism; Peptidoglycan biosynthesis; Penicillin and cephalosporin biosynthesis; Pantothenate and CoA biosynthesis; Nitrogen metabolism; Cyanoamino acid metabolism; Nicotinate and nicotinamide metabolism; Naphthalene degradation; Methane metabolism; Glycine metabolism; serine metabolism; threonine metabolism; Lysine degradation; Lipopolysaccharide biosynthesis; Inositol phosphate metabolism; Indole alkaloid biosynthesis; Histidine metabolism; Glycerolipid metabolism; Glycerophospholipid metabolism; Phosphatidylinositol signaling system; Fluorobenzoate degradation; Fatty acid biosynthesis; Dioxin degradation; Chlorocyclohexane and chlorobenzene degradation; Butanoate metabolism; Fatty acid metabolism; Arachidonic acid metabolism; Amino sugar metabolism; nucleotide sugar metabolism; vitamin B6 metabolism; geraniol degradation; cintronella degradation; limonene degradation; and pinene degradation.

Exemplary Method No. 39: The method of any one of Exemplary Method Nos. 32 to 38, wherein the determining of the first or second metabolome compound matrix comprises subjecting the gut sample to an analytical method selected from the group consisting of: a genomics method assessment, a transcriptomic or metabolomics assessment, a determination of microbiome composition, nuclear magnetic resonance (NMR) and mass spectrometry (MS), Fourier-transform infrared (FTIR), infrared (IR) thermography, cataluminescence (CTL), laser-induced fluorescence imaging (LIFT), and resonance-enhanced multiphoton ionization (REMPI).

Exemplary Method No. 40: The method of any one of Exemplary Method Nos. 32 to 39, wherein the determining of the first or second metabolome compound matrix is obtained from one or a plurality of subjects.

Exemplary Method No. 41: A method for identifying one or more biochemical pathways associated with a phenotype of a gut sample comprising: determining a first metagenome matrix of a gut sample of at least one subject lacking a phenotype; determining a second metagenome of a gut sample of at least one subject possessing a phenotype; comparing the first and second metagenome matrices; and associating differences between the first and second metagenome matrices to at least one biochemical pathway of the subject wherein the at least one biochemical pathway is associated with the phenotype.

Exemplary Method No. 42: The method of Exemplary Method No. 41, wherein the phenotype comprises insect repellence.

Exemplary Method No. 43: The method of Exemplary Method No. 41, wherein the phenotype comprises insect attraction.

Exemplary Method No. 44: The method of any one of Exemplary Method Nos. 41 to 43, wherein the phenotype comprises a gut metabolite.

Exemplary Method No. 45: The method of any one of Exemplary Method Nos. 41 to 44, wherein the gut sample is selected from the group consisting of: esophagus, stomach, small intestine, large intestine, and a fecal material.

Exemplary Method No. 46: The method of any one of Exemplary Method Nos. 41 to 45, further comprising identifying at least one agent that is a compound of the at least one biochemical pathway.

Exemplary Method No. 47: The method of any one of Exemplary Method Nos. 41 to 46, wherein the at least one biochemical pathway is selected from the group consisting of: Xylene degradation; Glucosinolate biosynthesis; Aminoacyl-tRNA biosynthesis; Penicillin and cephalosporin biosynthesis; Valine degradation; Leucine degradation; Isoleucine degradation; Tryptophan metabolism; Steroid biosynthesis; Starch and sucrose metabolism; Pyrimidine metabolism; Purine metabolism; Propanoate metabolism; Butanoate metabolism; Citrate cycle; Tyrosine metabolism; Phenylalanine metabolism; caprolactam degradation or metabolism; Toluene degradation; Glyoxylate metabolism; Dicarboxylate metabolism; Alanine metabolism; Aspartate metabolism; Glutamate metabolism; Propanoate metabolism; Porphyrin metabolism; Chlorophyll metabolism; Polycyclic aromatic hydrocarbon degradation; Benzoate degradation; Phosphonate metabolism; Phosphinate metabolism; Peptidoglycan biosynthesis; Penicillin and cephalosporin biosynthesis; Pantothenate and CoA biosynthesis; Nitrogen metabolism; Cyanoamino acid metabolism; Nicotinate and nicotinamide metabolism; Naphthalene degradation; Methane metabolism; Glycine metabolism; serine metabolism; threonine metabolism; Lysine degradation; Lipopolysaccharide biosynthesis; Inositol phosphate metabolism; Indole alkaloid biosynthesis; Histidine metabolism; Glycerolipid metabolism; Glycerophospholipid metabolism; Phosphatidylinositol signaling system; Fluorobenzoate degradation; Fatty acid biosynthesis; Dioxin degradation; Chlorocyclohexane and chlorobenzene degradation; Butanoate metabolism; Fatty acid metabolism; Arachidonic acid metabolism; Amino sugar metabolism; nucleotide sugar metabolism; vitamin B6 metabolism; geraniol degradation; cintronella degradation; limonene degradation; and pinene degradation.

Exemplary Method No. 48: The method of any one of Exemplary Method Nos. 41 to 47, wherein the determining of the first or second metagenome matrix is obtained from one or a plurality of subjects.

Exemplary Method No. 49: The method of any one of Exemplary Method Nos. 41 to 48, wherein the metagenome matrix comprises a metatranscriptome.

Exemplary Method No. 50: The method of any one of Exemplary Method Nos. 41 to 49, further comprising identifying at least one agent that modulates the gene expression of at least one component for the at least one biochemical pathway.

Exemplary Method No. 51: A method of repelling insects, comprising: topically applying on a subject a composition that modulates a skin microbiome biochemical pathway; and modulating the skin microbiome biochemical pathway to generate a compound that repel insects.

Exemplary Method No. 52: The method of Exemplary Method No. 51, wherein the subject is a human.

Exemplary Method No. 53: The method of Exemplary Method No. 51, wherein the insect is a mosquito.

Exemplary Method No. 54: The method of Exemplary Method No. 51, wherein the compound is octanoic acid, 1,4-dichlorobenzene, benzaldehyde, or naphthalene.

Exemplary Method No. 55: The method of Exemplary Method No. 51, wherein the composition is formulated as a spray, a spritz, a lotion, a cream, a sunscreen, an ointment, an oil, a solution, a vapor, an emollient, a paste, or a salve.

Exemplary Method No. 56: A method of treating, preventing, inhibiting, or ameliorating an oral disorder, comprising: administering to a subject a composition that modulates an oral microbiome biochemical pathway; and modulating the oral microbiome biochemical pathway to generate a compound that treats, prevents, inhibits, or ameliorates the oral disorder.

Exemplary Method No. 57: The method of Exemplary Method No. 56, wherein the subject is a human.

Exemplary Method No. 58: The method of Exemplary Method No. 56, wherein the oral disorder is dental caries or dental periodontitis.

Exemplary Method No. 59: The method of Exemplary Method No. 56, wherein the composition is formulated as an oral ingestible composition.

Exemplary Method No. 60: The method of Exemplary Method No. 59, wherein the oral ingestible composition is a lozenge, a powder, a pellet, a tables, chewable tables, a pill, a capsule, a solution, a drink, a paste, or a spray.

Exemplary Method No. 61: A method of treating, preventing, inhibiting, or ameliorating a skin disorder, comprising: administering to a subject a composition that modulates a skin microbiome biochemical pathway; and modulating the skin microbiome biochemical pathway to generate a compound that treats, prevents, inhibits, or ameliorates the skin disorder.

Exemplary Method No. 62: The method of Exemplary Method No. 61, wherein the subject is a human.

Exemplary Method No. 63: The method of Exemplary Method No. 61, wherein the skin disorder is atopic dermatitis.

Exemplary Method No. 64: The method of Exemplary Method No. 61, wherein the composition is formulated as a spray, a spritz, a lotion, a cream, a sunscreen, an ointment, an oil, a solution, a vapor, an emollient, a paste, or a salve for topical administration.

Exemplary Composition No. 65: A composition for modulating a microbiome in a subject, comprising a compound that modulates a microbiome biochemical pathway, wherein the composition is prepared by a process of: determining a first metagenome matrix of a microbiome of a subject lacking a target phenotype; determining a second metagenome of a microbiome of a subject possessing the target phenotype; comparing the first and second metagenome matrices; determining a compound that modulates the microbiome biochemical pathway by associating differences between the first and second metagenome matrices to at least one biochemical pathway of the subject wherein the at least one biochemical pathway is associated with the target phenotype; and preparing a composition comprising the compound that modulates the microbiome biochemical pathway.

Exemplary Composition No. 66: The composition of Exemplary Composition No. 66, wherein the microbiome is a gut microbiome, skin microbiome, lung microbiome, or oral microbiome.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural or singular terms herein, those having skill in the art can translate from the plural to the singular or from the singular to the plural as is appropriate to the context or application. The various singular/plural permutations may be expressly set forth herein for sake of clarity.

The embodiments illustratively described herein suitably may be practiced in the absence of any element(s) not specifically disclosed herein. Thus, for example, in each instance herein any of the terms “comprising,” “consisting essentially of,” and “consisting of” may be replaced with either of the other two terms. The terms and expressions which have been employed are used as terms of description and not of limitation, and use of such terms and expressions do not exclude any equivalents of the features shown and described or portions thereof, and various modifications are possible within the scope of the technology claimed. The term “a” or “an” can refer to one of or a plurality of the elements it modifies (e.g., “a reagent” can mean one or more reagents) unless it is contextually clear either one of the elements or more than one of the elements is described. The term “about” as used herein refers to a value within 10% of the underlying parameter (i.e., plus or minus 10%), and use of the term “about” at the beginning of a string of values modifies each of the values (i.e., “about 1, 2 and 3” refers to about 1, about 2 and about 3). For example, a weight of “about 100 grams” can include weights between 90 grams and 110 grams. Further, when a listing of values is described herein (e.g., about 50%, 60%, 70%, 80%, 85% or 86%) the listing includes all intermediate and fractional values thereof (e.g., 54%, 85.4%). Thus, it should be understood that although the present technology has been specifically disclosed by representative embodiments and optional features, modification and variation of the concepts herein disclosed may be resorted to by those skilled in the art, and such modifications and variations are considered within the scope of the embodiments.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

The entirety of each patent, patent application, publication and document referenced herein hereby is incorporated by reference. Citation of the above patents, patent applications, publications and documents is not an admission that any of the foregoing is pertinent prior art, nor does it constitute any admission as to the contents or date of these publications or documents. Their citation is not an indication of a search for relevant disclosures. All statements regarding the date(s) or contents of the documents is based on available information and is not an admission as to their accuracy or correctness.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1-50. (canceled)

51. A method of repelling insects, comprising:
topically applying on a subject a composition that modulates a skin microbiome biochemical pathway; and
modulating the skin microbiome biochemical pathway to generate a compound that repels insects.

52. The method of claim 51, wherein the subject is a human.

53. The method of claim 51, wherein the insect is a mosquito.

54. The method of claim 51, wherein the compound is octanoic acid, 1,4-dichlorobenzene, benzaldehyde, or naphthalene.

55. The method of claim 51, wherein the composition is formulated as a spray, a spritz, a lotion, a cream, a sunscreen, an ointment, an oil, a solution, a vapor, an emollient, a paste, or a salve.

56-66. (canceled)
